(19) United States
(12) Patent Application Publication
(10) Pub. No.: US 2009/0304654 A1
(43) Pub. Date: Dec. 10, 2009

(54) METHODS FOR ISOLATING ADIPOSE-DERIVED STEM CELLS AND THERAPEUTIC USE THEREOF

(75) Inventors: Tom F. Lue, Hillsborough, CA (US); Ching Shwun Lin, San Mateo, CA (US); Guiting Lin, San Francisco, CA (US); Maurice M. Garcia, San Francisco, CA (US); Peter R. Carroll, San Francisco, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: REGENTS OF THE UNIVERSITY OF CALIFORNIA, Oakland, CA (US)

(21) Appl. No.: 12/433,000

(22) Filed: Apr. 30, 2009

Related U.S. Application Data
(60) Provisional application No. 61/049,339, filed on Apr. 30, 2008.

(51) Int. Cl. A61K 45/00; C12N 5/10; A61P 3/10; A61P 9/10; C12N 9/08

(52) U.S. Cl. 424/93.21; 435/377; 435/381; 435/374; 435/192

(57) ABSTRACT

The application discloses adipose tissue-derived stem cells (ADSC) and related compositions and methods. ADSCs are useful for (i) production of insulin producing cells, (ii) treatment of diabetes, (iii) endothelial cell reconstitution, (iv) treatment of overactive bladder and urge incontinence, (v) prevention and treatment of bladder voiding dysfunction; (vi) treatment of neurogenic impotence such as that resulting from diabetes or after prostate cancer therapy; (vii) treatment of vasculogenic impotence, such as that resulting from hypertension, dyslipidemia, atherosclerosis and diabetes; (viii) the promotion of wound healing; (ix) reduction of skin wrinkling; and (x) hair growth.

### CROSS-REFERENCES TO RELATED APPLICATIONS

This application claims the benefit of U.S. Provisional Application Ser. No. 61/049,339 filed Apr. 30, 2008, the contents of which are hereby incorporated by reference in its entirety.

### STATEMENT AS TO RIGHTS TO INVENTIONS MADE UNDER FEDERALLY SPONSORED RESEARCH OR DEVELOPMENT

This invention was supported in part by funds from the U.S. government (NIDDK Grant Nos. P50Dk64538 and R37 DK045370) and the U.S. government has certain rights in the invention.

### BACKGROUND OF THE INVENTION

The adipose tissue contains a stromal vascular fraction (SVF) from which multipotent cells have been isolated. These cells are variously called processed lipoaspirate (PLA) cells, adipose tissue-derived mesenchymal stem cells, multipotent adipose-derived stem (MADS) cells, adipose tissue-derived stem cells, adipose tissue-derived stromal cells (ADSC, ATSC), adipose tissue derived adult stem (ADAS) cells, adipose tissue-derived adult stromal (ADAS) cells, and adipose tissue-derived cells (ADC).

ADSC possess phenotypes and gene expression profiles similar to those of bone marrow stem cells (BMSC). In addition to having the capacity for self-renewal and long-term growth, ADSC are capable of differentiating into diverse cell types including adipocytes, osteoblasts, chondrocytes, hepatocytes, myocytes, cardiomyocytes, neurons, and epithelial cells. Thus, ADSC are not only increasingly accepted as bona fide adult stem cells but also considered to be superior to other types of adult stem cells for future clinical applications. Whereas bone marrow can only be obtained in limited quantity because of donor site morbidity, the adipose tissue is usually obtainable in abundance, especially in our increasingly obese society. In addition, clonogenic studies have established that the number of BMSC in bone marrow is approximately 1 in 25,000 to 1 in 100,000, whereas the average frequency of ADSC in processed lipoaspirate is approximately 2% of nucleated cells. Thus, the yield of ADSC from 1 g of fat is approximately 5000 cells, whereas the yield of BMSC is 100-1000 cells per milliliter of marrow.

The present invention provides improved methods for culturing, isolating and modifying populations of particularly useful adipose tissue-derived stem cells (ADSC), as well as novel methods for treating human subjects with ADSC.

### BRIEF SUMMARY OF THE INVENTION

In some embodiments, the invention provides a method for generating Insulin-Producing Adipose tissue Derived Stem Cells (IPADSC), said method comprising the steps of isolating a population of ADSC and introducing an expression cassette driving expression of Pdx-1 or VP16 into said population of ADSC, thereby generating IPADSC. In some embodiments, the ADSC are human cells. In some embodiments, the expression cassette is introduced in a lentiviral vector or liposomal vector. In some embodiments, the method further comprises administering the IPADSC to a subject, e.g., by injection or by surgical transplant. In some embodiments, the subject has diabetes (type 1 or type 2), or has a predisposition or risk for diabetes.

In some embodiments, the invention a method of treating or preventing diabetes in a subject, said method comprising administering a composition comprising IPADSC to the subject, thereby treating or preventing diabetes in the subject. In some embodiments, the composition comprises as least 10⁶ IPADSC, e.g., at least 5×10⁶, 10⁷, 5×10⁷, 10⁸, 5×10⁸, or 10⁹ IPADSC. In some embodiments, the administration is by injection or surgical transplant. In some embodiments, the IPADSC are syngeneic or autologous. In some embodiments, the administration is repeated at least once. In some embodiments, the subject has type 1 diabetes. In some embodiments, the subject has type 2 diabetes, or has a predisposition for diabetes. In some embodiments, the subject has age-related decline in organ function. In some embodiments, the method further comprises monitoring the subject, e.g., for symptoms of diabetes or reduced organ function, such as high fasting blood glucose.

The invention provides methods of generating Endothelial-like ADSC (EADSC), said method comprising contacting ADSC with media comprising Fibroblast Growth Factor 2 (FGF2). In some embodiments, the media further comprises Vitamin C. In some embodiments, the ADSC are cultured in said media for at least 3 days, e.g., 4, 5, 6, 7, or 8 days. In some embodiments, the method further comprises administering the EADSC to a subject, e.g., by injection or by surgical transplant. In some embodiments, the subject has a hypoxia- or ischemia-related disorder, or age-related or neuropathic decline in tissue function.

In some embodiments, the invention provides a method for treating or preventing a hypoxia- or ischemia-related disorder (or similar age-related or neuropathic decline in tissue function) in a subject, comprising contacting ADSC with media comprising FGF2, such that the ADSC are transformed into EADSC; and administering a composition comprising said EADSC to said subject, thereby treating or preventing the hypoxia- or ischemia-related disorder in the subject. In some embodiments, the media further comprises Vitamin C. In some embodiments, the composition comprises as least 10⁶ EADSC, e.g., at least 5×10⁶, 10⁷, 5×10⁸, 5×10⁸, or 10⁹ EADSC. In some embodiments, the administration is by injection or surgical transplant. In some embodiments, the EADSC are syngeneic or autologous. In some embodiments, the administration is local, e.g., to the site of the ischemia or hypoxia. In some embodiments, the administration is repeated at least once. In some embodiments, said ischemia-related disorder is selected from the group consisting of stroke, angina, bed ulcer, foot ulcer, intermittent claudication and kidney disease.

In some embodiments, the invention provides methods for treating or preventing peripheral neuropathy in a subject, said method comprising administering a composition comprising Adipose tissue Derived Stem Cells (ADSC) to the subject, thereby treating or preventing peripheral neuropathy in the subject. In some embodiments, the composition comprises as least 10⁶ ADSC, e.g., at least 5×10⁶, 10⁷, 5×10⁸, 5×10⁸, or 10⁹ ADSC. In some embodiments, the administration is by injection, e.g., local injection to a site of reduced sensation. In some embodiments, administration is systemic. In some embodiments, the ADSC are syngeneic or autologous. In some embodiments, the subject has diabetes, or age-related decline in organ function. In some embodiments, the ADSC are multipotent ADSC isolated as disclosed herein.

In some embodiments, the invention provides methods of treating urinary tract injury or incontinence in a subject, said method comprising administering a composition comprising Adipose tissue Derived Stem Cells (ADSC) to the subject, thereby treating the urinary tract injury or incontinence in the subject. In some embodiments, the composition comprises as least 10⁶ ADSC, e.g., at least 5×10⁶, 10⁷, 5×10⁷, 10⁸, 5×10⁸, or 10⁹ ADSC. In some embodiments, administration is by injection or surgical implantation into the bladder and/or urethra of said subject. In some embodiments, the ADSC are syngeneic or autologous. In some embodiments, the urinary tract injury is a result of pregnancy or birth. In some embodiments, the subject is diabetic. In some embodiments, the ADSC are multipotent ADSC isolated as disclosed herein.

In some embodiments, the invention provides methods for treating erectile dysfunction or impotence in a subject, said method comprising administering a composition comprising Adipose tissue Derived Stem Cells (ADSC) to the penis of the subject, thereby treating erectile dysfunction or impotence in the subject. In some embodiments, the composition comprises as least 10⁶ ADSC, e.g., at least 5×10⁶, 10⁷, 5×10⁷, 10⁸, 5×10⁸, or 10⁹ ADSC. In some embodiments, the subject has neurogenic impotence. In some embodiments, the administration is by injection to the corpus cavernosum. In some embodiments, the cells are multipotent ADSC isolated as disclosed herein.

In some embodiments, the invention provides methods for treating a wound in a subject, said method comprising administering a composition comprising Adipose tissue Derived Stem Cells (ADSC) to the wound area, thereby treating the wound in the subject. In some embodiments, the composition comprises as least 10⁶ ADSC, e.g., at least 5×10⁶, 10⁷, 5×10⁷, 10⁸, 5×10⁸, or 10⁹ ADSC. In some embodiments, the administration is by subcutaneous or parenteral injection. In some embodiments, the composition is administered to the site of a surgical wound. In some embodiments, the cells are multipotent ADSC isolated as disclosed herein.

In some embodiments, the invention provides a method for restoring hair growth in a subject, comprising the local injection of a composition comprising adipose tissue-derived stem cells into the area where hair follicle structures are not functional or have been damaged, e.g., by radiation, injuries or aging. In some embodiments, the cells are multipotent ADSC isolated as disclosed herein.

In some embodiments, method for replenishing gonadal cells in a subject, comprising injection of ADSCs (harvested from adipose tissue from the subject and used autologously or provided by a donor) into the testicle(s)/gonads of the subject. Non-limiting examples of gonadal cells include spermatogonial cells, Leydig cells, sertoli cells and intratesticular micro-vascular cells such as endothelial cells.

In some embodiments, the invention provides a method for treating wrinkles or soft tissue defect in a subject, comprising the subcutaneous injection of a composition comprising adipose tissue-derived stem cells at the site of said wrinkles or soft tissue defect. In some embodiments, the cells are multipotent ADSC isolated as disclosed herein.

Those of skill in the art will recognize that the ADSC utilized for therapeutic methods and compositions can be isolated and/or cultured and/or maintained according to any of the methods disclosed herein.

In some embodiments, the invention provides a method for isolating adipose tissue-derived stem cells, comprising: (i) incubating a solution containing adipose tissue and collagenase; (ii) separating the lipids, collagenase and adipose cells in the incubated solution into layers by subjecting the incubated solution to centrifugation; (iii) isolating and filtering the collagenase layer; (iv) subjecting the filtered collagenase layer to a second centrifugation step, thereby obtaining a preparation of recycled collagenase; and (v) treat fresh whole-resected adipose tissue, or liposuction-removed (lipoaspirate) with said recycled collagenase.

In some embodiments, said recycled collagenase is utilized at a higher collagenase: adipose ratio in step (v) than the collagenase: adipose ratio utilized in step (i). In another embodiment, said collagenase: adipose ratio in step (i) is at least 2:1 volume/volume, assuming a 0.075% starting solution of collagenase. In another embodiment, said collagenase is type 1A collagenase. In some embodiments, collagenase is “recycled” for reuse in samples from the same individual. Collagenase is a durable (and expensive) enzyme, and can be used to digest several fat samples from an individual (but not other individuals).

In some embodiments, the invention provides a method for preserving a sample of adipose cells, adipose tissue, or adipose tissue-derived stem cells, comprising: (i) combining said sample with a preservation solution comprising superoxide dismutase at a concentration of at least 5,000 U/L; and (ii) mixing said solution with said sample.

In some embodiments, said preservation solution further comprises at least one additional pharmaceutically compatible substance selected from the group consisting of potassium phosphate, potassium chloride, sodium phosphate, human serum albumin, and sodium chloride.

In some embodiments, the invention provides a composition comprising cells selected from the group consisting of isolated adipose cells, adipose tissue, and adipose tissue-derived stem cells; and (ii) superoxide dismutase, wherein said superoxide dismutase is present at a concentration of at least 5,000 U/L.

In some embodiments, the invention provides a method for isolating a subpopulation of multipotent adipose tissue-derived stem cells, comprising: (i) contacting a starting population of isolated adipose tissue-derived stem cells (ADSC) with a set of antibodies, wherein said set of antibodies comprises anti-CD34, anti-CD90, and anti-SSEA1 (stage specific embryonic antigen 1) antibodies; (ii) identifying the adipose tissue-derived stem cells which express the antigens recognized by said set of antibodies; and (iii) isolating the adipose tissue-derived stem cells identified in step (ii), wherein the isolated cells constitute a multipotent population of adipose tissue-derived stem cells. In some embodiments, the identifying step is carried out by a detection device or computer. ADSC are multipotent before subfractionation.

In some embodiments, the invention provides a therapeutic, injectable adipose tissue-derived stem cell composition comprising a mixture of adipose derived stem cells, carboxymethylcellulose, and 60 μm poly(lactic-co-glyocolyic acid) microspheres. In some embodiments, the ratio of said stem cells to said microspheres is less than 1:10, e.g., 1:12, 1:15, 1:20, 1:50, or 1:100, etc.

In some embodiments, the invention provides a method for growing adipose tissue-derived stem cells (ADSC), comprising incubating said ADSC in a serum-free solution comprising at least 2% platelet lysate. In some embodiments, the solution comprises at least 3%, 4%, 5%, 6%, 7%, 8%, 9%, 10% or more platelet lysate. In some embodiments, the platelets are from a human.

In some embodiments, the invention provides a method for preparing adipose tissue-derived stem cells (ADSC) for therapeutic administration, comprising (i) incubating a solution containing adipose tissue and collagenase; (ii) separating the lipids, collagenase and adipose cells in the incubated solution into layers by subjecting the incubated solution to centrifugation; (iii) isolating and filtering the collagenase layer; (iv) subjecting the filtered collagenase layer to a second centrifugation step, thereby obtaining a preparation of recycled collagenase; (v) treating fresh adipose tissue of lipoaspirate with said recycled collagenase; (vi) isolating said ADSC by centrifugation; (vii) culturing said adipose cells on a culture dish; (viii) identifying adipose tissue-derived stem colonies arising from step (vii); (ix) culturing the ADSC from the colonies identified in step (viii) in a serum-free media comprising at least 2% platelet lysate; and (x)isolating adipose tissue-derived stem cells obtained from the culture of step (ix) and mixing the isolated adipose tissue-derived stem cells with 60 μm poly(lactic-co-glyocolyic acid) microspheres, wherein said ratio of stem cells to microspheres is less than 1:1.

In some embodiments, the invention provides method of operating an adipose tissue derived stem cell bank, the methods comprising the steps of: (a) removing adipose tissue of a human subject by liposuction or surgical excision; (b) isolating said adipose tissue-derived stem cells according to at least one of the methods described above; (c) storing the cells in the solution described above and freeze storing in liquid nitrogen for future use.

### BRIEF DESCRIPTION OF THE DRAWINGS

FIG. 1: FIG. 1A shows the results of in vivo injections of BrdU-labeled ADSC mixed within Matrigel, viewed at 400×. The arrow marked “A” depicts a blood vessel; arrow “B” shows the red blood cells (disc shaped) within the blood vessel; arrow “C” shows BrdU-labeled ADSC. Note that the ADSC are incorporated into the wall of the blood vessel, suggesting that they contributed to its formation. FIG. 1B depicts another section following the same procedure as that used in FIG. 1B. The “A” arrows point to skeletal muscle tissue (large arrow) and a skeletal muscle cell nucleus (small arrow). Note that normal nuclei (non-ADSC derived) stain darker (i.e., staining with hematoxylin and eosin or “H & E”). Arrow “B” shows the BrdU-positive nucleus of a skeletal muscle cell, indicating that was derived from an ADSC. Arrow “C” is drawn to a blood vessel. Arrow “D” points to ADSC-derived cells present within the walls of the blood vessel. Arrow “E” points to red blood cells within the lumen of the blood vessel. FIG. 1C shows the results of an experiment in which rat ADSC were suspended in Matrigel, then injected (1 ml) subcutaneously into loose skin in the rat's shoulder area. After 10 days, the Matrigel-injected area was harvested, sectioned, and stained with H & E and for BrdU. Arrow “A” points to BrdU labeled ADSC. The ADSC form tube structures (B.) that resemble blood vessels, which contain Red Blood Cells (C.) within their lumen.

FIG. 2 depicts the results of experiments demonstrating the efficacy of treating surgical wounds with ADSC. Injection site tissues were resected 4 weeks after injection of Sprague-Dawley rats. FIG. 2A shows an image from a control, at 200× magnification, where a rat's wound incision was treated by a sham injection into the subcutaneous space. The black arrow points to an H & E stained nucleus. There is no BrdU staining of the cells (shaded arrow). FIG. 2B shows an image from the same experiment as that described in FIG. 2A, except at higher (400×) magnification. The black segment of the “A” arrow points to skeletal muscle fiber bundles with nuclei exhibiting the normal H & E staining, and the shaded segment of the “A” arrow shows the absence of BrdU staining of the cells. Arrow “B” points to a blood vessel (two crescentric-shaped cells forming circular pattern) exhibiting typical H & E staining. FIG. 2C shows an image of a tissue section taken from a surgical wound incision that was treated by injecting BrdU-labeled ADSC into the subcutaneous space. Arrow “A” points to a blood vessel. Arrow “B” points to a red blood cell within the blood vessel. Arrow “C” points to an ADSC nucleus staining positive for BrdU. The arrow in “D” point to connective tissue, and the arrow “E” points to BrDU-positive cells within the connective tissue, which resemble skeletal muscle cells and suggest that these muscle cells were derived from ADSC. FIG. 2D shows another image from the same experiment as FIG. 2C. Arrow “A” points to a blood vessel with BrdU-stained ADSC nuclei. Arrow “B” points to skeletal muscle with BrdU-stained nuclei indicative of differentiation from ADSC.

FIG. 3 depicts a comparison between bladder wall treated by a sham injection (vehicle-only Control; left panels) versus injection of ADSC, at two different magnifications. The tissue specimens were stained with Rat Endothelial Cell Antibody (RECA), a marker for blood vessel endothelial cells. The samples were taken four weeks after injection. The figure shows that ADSC-treated tissue shows more RECA-positive staining than the control indicating that ADSCs have the ability to stimulate formation of new blood vessels.

FIG. 4 depicts variations in the induction of CD31 expression in rat ADSC (RADSC) versus rat urethra smooth muscle cells (RUSMC). RADSC and RUSMC were cultured in DMEM or endothelial growth medium (EGM2). As a control, human umbilical vein endothelial cells (HUVEC) were cultured in EGM2. Immunofluorescence staining for CD31 shows an increase of the number of CD31-positive cells in RADSC cultured in EGM2 when compared to RADSC cultured in DMEM. In contrast, the number of CD31 -positive cells RUSMC was similarly low in both DMEM and EGM2. The figure shows that ADSC can be directed by local tissues to become endothelial-like cells.

FIG. 5 depicts tube formation by RADSC cell lines cultured in the presence (FIG. 5A) and absence (FIG. 5B) of HUVEC. To achieve the results shown in FIG. 5A, two RADSC cell lines (RADSC-1 and RADSC-2) and RUSMC were labeled with DiI and each was mixed with calcein AM-labeled HUVEC at a ratio of 1:4. The cells were then cultured in Matrigel for 16 h. To achieve the results shown in FIG. 5B, the RADSC-1, RADSC-2, and RUSMC cell lines were labeled with DiI and cultured in Matrigel for 16 h (top panels, visualized by fluorescence microscopy) or 10 days (lower panels, visualized by phase contrast microscopy). The figure shows that ADSC can be directed by local tissues to become endothelial-like cells.

FIG. 6 shows human ADSCs transfected with Lenti-GFP, Lenti-PDX1, or Lenti-VP16, and cultured in differentiation medium for 21 days. The morphology of GFP-transfected cells remained unchanged while that of PDX1- or VP16-transfected cells changed significantly. VP16 is an engineered version of PDX1. GFP is a green fluorescence protein, serving as a negative control.

FIG. 7 shows PDX1 expression in HADSCs transfected by lentivirus. The left panel displays the results of PDX1 mRNA expression by RT-PCR. mPDX1 denotes mouse PDX1, which was transfected into human ADSC cell lines 20 and 24. hPDX1 denotes human PDX1. Right panel displays the results of PDX1 protein expression by western blot analysis.

FIG. 8 shows the quantification of insulin production (u/ml) by transfected ADSCs as measured by ELISA.

FIG. 9 shows verification of Pdx1 and insulin expression. Human and rat ADSC were transduced with GFP (control), Pdx1 or Pdx1-VP16 (PV16). Expression of Pdx1 in these cells was examined by western blotting (with β-actin serving as control, Panel A) and RT-PCR (Panel B). Static insulin production by human ADSC (in DMEM with 23 mM glucose) was further examined by ELISA (Panel C).

FIG. 10 shows pancreatic gene expression in IPADSC (Insulin Producing ADSC). Human and rat ADSC were untransduced (C) or transduced with GFP or Pdx1. These cells and rat urethra smooth muscle cells (RUSMC) were examined by RT-PCR for the expression of Pdx1, insulin, glucagon, and NeuroD (with b-actin serving as control, Panel A). Statistical analyses of the results for the human and rat cells are presented in Panels B (n=3) and C (n=5), respectively. Asterisks indicate significant differences (P<0.05) between Pdx1-transduced cells and untransduced cells.

FIG. 11 shows insulin production in response to glucose concentration. Pdx1-transduced cells were incubated in buffer containing the indicated concentrations of glucose. One hour later the amount of insulin in the buffer was assessed by ELISA. Asterisks indicate significant differences (P<0.05) as compared to insulin production at 0 mM of glucose.

FIG. 12 shows changes in fasting blood glucose levels and body weight. Thirty rats were randomly and equally divided into three groups. The first group (Control) received intraperitoneal injection of 20 mM citrate buffer. The second and third groups both received intraperitoneal injection of 60 mg of STZ (in 20 mM citrate buffer) per kg of body weight. One week later the second group (Saline) received saline treatment while the third group (IPADSC) received IPADSC treatment. Asterisks indicate significant differences (P<0.05) between IPADSC-treated and saline-treated rats.

FIG. 13 shows glucose tolerance at the end of the seven week study. Rats fasted for seven hours received intraperitoneal injection of 1 mg of glucose per gram of body weight. Blood glucose levels were then monitored for 2 h at 30-min intervals in samples obtained from the tail vein. Asterisks indicate significant differences (P<0.05) between IPADSC-treated and saline-treated rats.

FIG. 14 shows the transplanted cells. At the end of the seventh week post-treatment, rats were sacrificed and their kidneys harvested for histological examination. HE staining was used to examine the subcapsular space for the presence of transplanted cells. Immunofluorescence (IF) staining was used to identify cells expressing insulin. Boxed areas in the 20× photos are injection sites and are enlarged in the respective 100× photos. The boxed areas in the 100× photos are further enlarged in the respective 400× photos. Note the tissue-like structures in the subcapsular space of the IPADSC-treated kidney. No such structure was visible in the saline-treated kidney. The IF photos were taken from 3 IPADSC-treated kidneys. Note the presence of insulin-positive cells

FIG. 15 shows the higher voiding pressure in ADSC-treated animals.

FIG. 16 shows co-localization of EdU and SMA. The red signal is EdU, green is ASMA, and blue is DAPI. The boxed area in each picture in the upper panels is shown in the corresponding picture in the lower panels (×400).

FIG. 17 shows elastic fibers in the urethra. Left: control. Right: ADSC transplanted (×400).

FIG. 18 shows the effect of ADSC treatment on ICP in ZDF rats.

FIG. 19 shows endothelial differentiation of ADSC in the penis. ADSC were labeled with BrdU and injected into the corpus cavernosa of rats. Four weeks later the tissues were examined by immunofluorescence microscopy. Anti-BrdU and RECA-1 antibodies identified the injected ADSC (green) and endothelial cells (red), respectively. Superimposed image (BrdU/RECA) shows that some ADSC (yellow) also stained positive for RECA-1. Another superimposed image (Merge) with the phase-contrast image shows the localization of ADSC to the sinusoid endothelium. Approximately 5% of BrdU+ cells were RECA+, as determined by counting 10 randomly selected areas in the cross section.

FIG. 20 shows comparison of cell morphology and growth rate in DMEM and EGM2. Two rat ADSC lines, RADSC-1 and RADSC-2, were seeded into 100-mm dishes at identical density (300,000 cells/dish) and grown for 3 days in DMEM or EGM2. The cell morphology of RADSC-1 is shown in panel A. The growth rate of both cell lines is shown in panel B

FIG. 21 demonstrates identification of endothelial-inducing factor by subtraction. RADSC-1 cells were grown in fully or partially supplemented EGM2 and then assayed for LDL-uptake. Each partially supplemented EGM2 is indicated by the omitted factor; for example, “−FGF” denotes EGM2 without FGF2. Experiments were repeated 3 times.

FIG. 22 demonstrates identification of endothelial-inducing factor by addition. RADSC-1 cells were grown in EGM2, EBM2, or EBM2 supplemented with the indicated factor, and then assayed for LDL-uptake. Experiments were repeated 3 times.

FIG. 23 shows the effect of FGFR inhibitor on endothelial differentiation. RADSC-1 cells were grown in EGM2 or EBM2 supplemented with FGF2 and vitamin C in the presence or absence of FGFR inhibitor PD173074. The cells were then assayed for LDL-uptake. Red color indicates the presence of LDL, which was in a conjugated form with the red fluorescence dye DiI. RADSC-1 cells were also grown in VEGF/vitamin C-supplemented EBM2 for the purpose of excluding the involvement of VEGF signaling, as PD173074 is known to have a weak inhibitory effect on VEGF receptor. Experiments were repeated 3 times.

FIG. 24 shows results of a Randall-Selito test. Lean mildly diabetic ZDF male rats age 13 weeks served as the control group. Animals labeled D-Non-treated were severely diabetic obese ZDF rats; baseline and 6 weeks after treatment measurements are shown. Animals labeled D-Treated correspond to severely diabetic obese ZDF rats that were treated with ADSC. All animals used in this study were of the same age.

FIG. 25 shows results of the Von-Frei Hair test. Lean mildly diabetic ZDF rats were labeled control group animals, now age 19 weeks. The animals labeled Diabetic were untreated severely diabetic obese ZDF animals. The animals labeled Tr (Diabetic) were severely diabetic obese ZDF animals treated with ADSC.

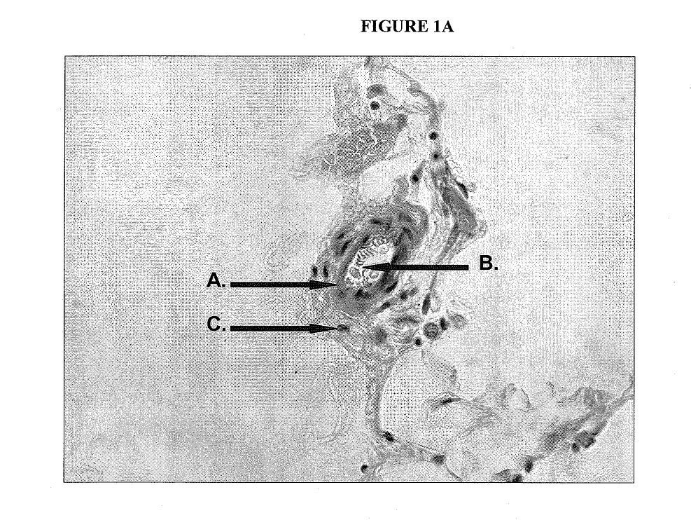

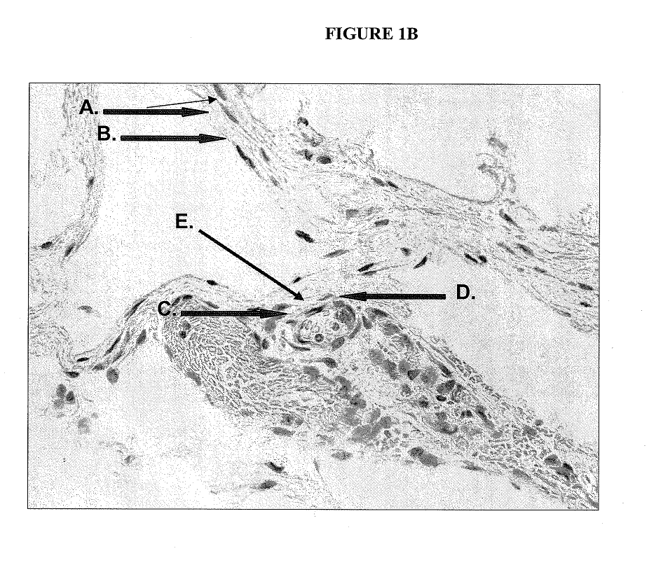

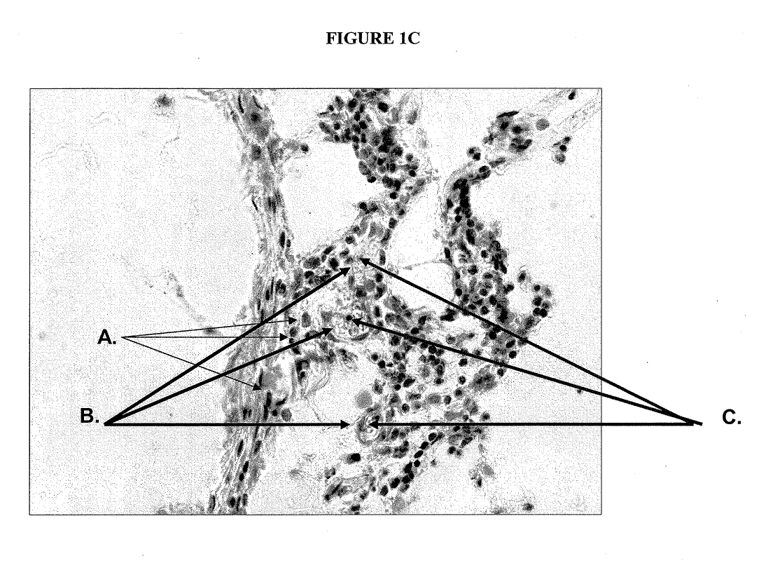

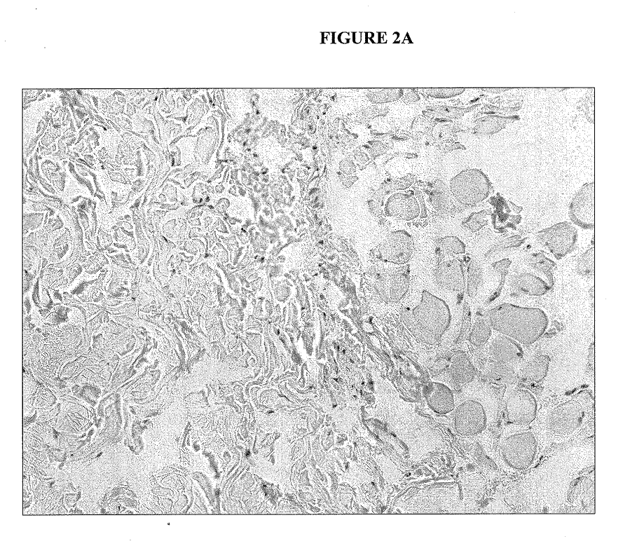

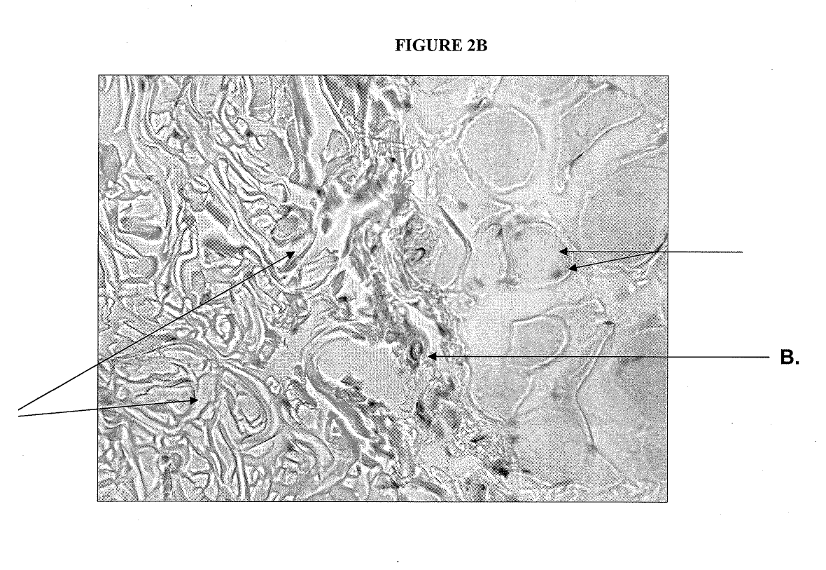

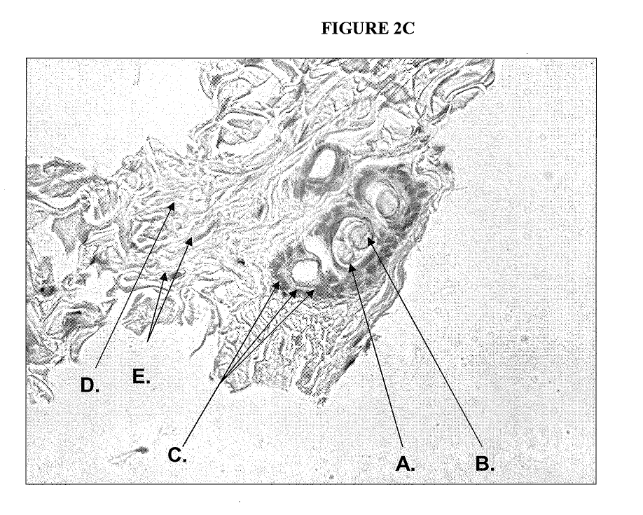

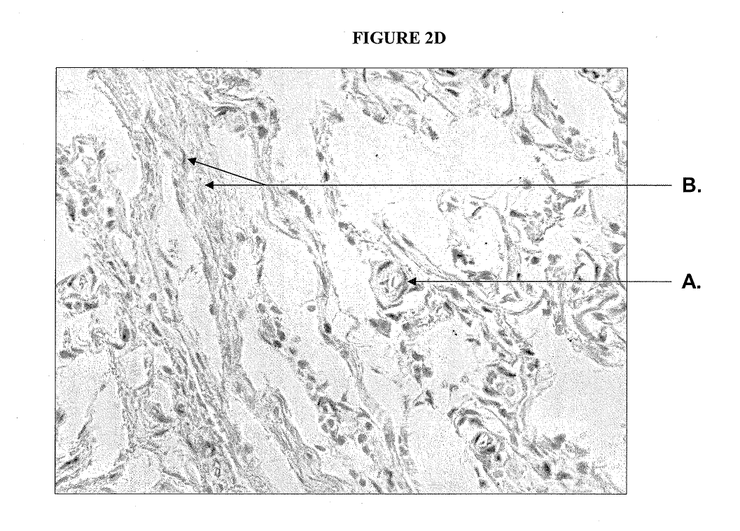

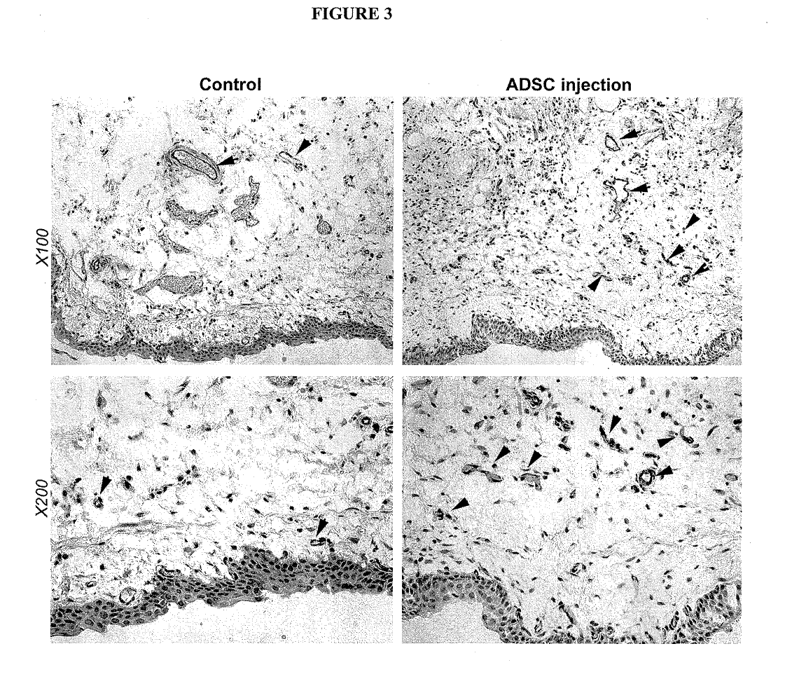

### DETAILED DESCRIPTION OF THE INVENTION

The present invention provides methods of isolating adipose-derived stem cells (ADSC) and methods for using the ADSC in treating various pathologies including, without limitation, diabetes, tissue regeneration, wound healing, scarring, soft tissue defect, impotence, overactive bladder, incontinence and hair loss in a mammal. ADSCs may also be administered for cosmetic purposes, including breast and penile enhancement, the removal of wrinkles, and similar applications where a change in the size and shape of a body feature is desired.

### 1. Definitions

The term “mammal” as used herein, encompasses any mammal. Preferably a mammal is in need of such treatment or prevention. Examples of mammals include, but are not limited to, cows, horses, sheep, pigs, cats, dogs, mice, rats, rabbits, guinea pigs, monkeys, etc., more preferably, a human.

“Adipose-derived stem cells” refers to multipotent stromal cells or stem cells that originate from adipose tissue and are capable of self-renewal. By “adipose” is meant any fat tissue. The adipose tissue may be brown or white adipose tissue, derived from subcutaneous, omental/visceral, mammary, gonadal, or other adipose tissue site. Preferably, the adipose is subcutaneous white adipose tissue. Such cells may comprise a primary cell culture or an immortalized cell line. The adipose tissue may be from any organism having fat tissue. Preferably, the adipose tissue is mammalian, most preferably the adipose tissue is human, and especially preferred is adipose tissue derived from the subject to be treated (i.e., autologous tissue) or a clone of the subject. These cells express a unique combination of cell surface proteins that can include, but are not limited to the tetraspan protein CD9, CALLA (CD10), aminopeptidase N (CD13), integrin 1 (CD29), hyaluronate receptor (CD44), integrin alpha. 4 and 5 (CD49d, CD49e), ICAM-1 (CD54), decay accelerating factor (CD55), complement protectin (CD59), endoglin (CD105), VCAM-1 (CD106), Muc-1,8 (CD 146), and ALCAM (CD166) (Gronthos et al. J. Cell Physiol. (2001) October; 189(1):54 63).

A “biological sample” can be obtained from a patient, e.g., tissue removed during surgery, e.g., liposuction; a biopsy, e.g., from an animal, such as an animal model; or from cultured cells, e.g., a cell line or cells removed from a patient and grown in culture for observation. Biological samples include tissue, such as adipose tissue, or bodily fluids, e.g., blood, blood fractions, lymph, saliva, urine, feces, etc.

As used herein, the terms “treat” and “prevent” are not intended to be absolute terms. Treatment can refer to any delay in onset, amelioration or reduction of symptoms, healing, improvement in patient health or survival, etc. The effect of treatment can be compared to an individual or pool of individuals not receiving the treatment, or to an untreated tissue in the same patient.

The term “autologous” refers to cells or tissues that are transplanted into the same individual from which they are drawn. Cells from a different individual can be referred to as allogeneic. The terms “syngenic” or “syngeneic” refer to genetically identical cells or tissue, or sufficiently identical and immunologically compatible, as to allow for transplantation. For example, syngeneic cells can be obtained for an individual from his or her identical twin. In the case of animals, syngeneic cells can be obtained from members of the same inbred strain.

Diabetes mellitus can be divided into two clinical syndromes, Type 1 and Type 2 diabetes mellitus. As used herein, “type 1,” “type I,” and “insulin-dependent diabetes mellitus (IDDM)” are used interchangeably. Type 1 diabetes is a chronic autoimmune disease characterized by the extensive loss of beta cells in the pancreatic Islets of Langerhans, which produce insulin. As these cells are progressively destroyed, the amount of secreted insulin decreases, eventually leading to hyperglycemia (abnormally high level of glucose in the blood) when the amount of secreted insulin drops below the level required for euglycemia (normal blood glucose level).

Type 2 diabetes (also referred to herein as “type II” and “non-insulin dependent diabetes mellitus (NIDDM)”) is a complex disease characterized by defects in glucose and lipid metabolism. Typically there are perturbations in many metabolic parameters including increases in fasting plasma glucose levels, free fatty acid levels and triglyceride levels, as well as a decrease in the ratio of HDL/LDL. The period of insulin resistance that characterizes Type II diabetes and which precedes the late stage decrease in insulin in this population, is recognized to be especially harmful to tissue and organ function. One application of autologous ADSC therapy is that ADSC can modulate insulin resistance, thereby improving tissue utilization of glucose, and thus reducing hyperglycemia and its systemic harmful effects.

“Insulin sensitivity” refers to effect of insulin on glucose uptake in a cell. Sensitivity can be determined at an organismal, tissue or cellular level. For example, blood or urine glucose levels following a glucose tolerance test are indicative of insulin sensitivity. Other methods of measuring insulin sensitivity include, e.g., measuring glucose uptake (see, e.g., Garcia de Herreros, A., and Birnbaum, M. J. J. Biol. Chem. 264, 19994-19999 (1989); Klip, A., Li, G., and Logan, W. J. Am. J. Physiol. 247, E291-296 (1984)), measuring the glucose infusion rate (GINF) into tissue such as the skeletal muscle (see, e.g., Ludvik et al., J. Clin. Invest. 100:2354 (1997); Frias et al., Diabetes Care 23:64, (2000)) and measuring sensitivity of GLUT4 translocation (e.g., as described herein) in response to insulin.

“Predisposition for diabetes” occurs in a person when the person is at high risk for developing diabetes. A number of risk factors are known to those of skill in the art and include: genetic factors (e.g., carrying alleles that result in a higher occurrence of diabetes than in the average population or having parents or siblings with diabetes); overweight (e.g., body mass index (BMI) greater or equal to 25 kg/m²); habitual physical inactivity, race/ethnicity (e.g., African-American, Hispanic-American, Native Americans, Asian-Americans, Pacific Islanders); previously identified impaired fasting glucose or impaired glucose tolerance, hypertension (e.g., greater or equal to 140/90 mmHg in adults); HDL cholesterol greater or equal to 35 mg/dl; triglyceride levels greater or equal to 250 mg/dl; a history of gestational diabetes or delivery of a baby over nine pounds; and/or polycystic ovary syndrome. See, e.g., “Report of the Expert Committee on the Diagnosis and Classification of Diabetes Mellitus” and “Screening for Diabetes” Diabetes Care 25(1): S5-S24 (2002).

“Ischemia-related disorders” include those conditions that result from restricted blood supply. The term “hypoxia-related disorder” is broader, referring to disorders resulting from a restricted oxygen supply. Ischemia can result due to damage to the blood vessels from injury or disease. Conditions related to ischemia include but are not limited to atherosclerosis, poor circulation (e.g., as observed in diabetic patients), hypotension, blood clots, embolisms, frost bite or localized extreme cold, injury, and endothelial cell dysfunction. Endothelial cell dysfunction can lead to a cascade of negative effects that include vessel clots, inflammation, atherosclerosis, and dysregulation of vessel contraction/dilation.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody or its functional equivalent will be most critical in specificity and affinity of binding. See Paul, Fundamental Immunology (2003).

### II. Adipose-Derived Stem Cells (ADSC) and Their Isolation

Adipose tissue offers a source of multipotent stromal cells. Adipose tissue is readily accessible and abundant in many individuals. Obesity is a condition of epidemic proportions in the United States, where over 50% of adults exceed the recommended BMI based on their weight and height. Adipocytes can be harvested by liposuction on an outpatient basis. This is a relatively non-invasive procedure with cosmetic effects that are acceptable to the vast majority of patients. It is well documented that adipocytes are a replenishable cell population. Even after surgical removal by liposuction or other procedures, it is common to see a recurrence of adipocytes in an individual over time. This suggests that adipose tissue contains stromal stem cells that are capable of self-renewal.

Methods for the ordinary isolation, expansion, and differentiation of human adipose tissue-derived stem cells have been described previously (Zuk et al., Tissue Engerineering (2001) 7:211-228; Burris et al Mol Endocrinol 1999, 13:410 7; Erickson et al Biochemical & Biophysical Research Communications 2002, 290:763 9; Gronthos, et al. J. Cell Physiol. October 2001;189(1):54 63; Halvorsen, et al, Metabolism 2001, 50:407 413; Halvorsen, et al, Tissue Eng. December 2001;7(6):729 41; Harp, et al. Biochem Biophys Res Commun 2001, 281:907 912; Saladin et al 1999, Cell Growth & Diff 10:43 48; Sen, et al. Journal of Cellular Biochemistry 2001, 81:312 319; Zhou et al Biotechnol Techniq 1999, 13:513 517). Adipose tissue-derived stem cells are obtained from minced human adipose tissue by collagenase digestion and differential centrifugation according to known techniques (Halvorsen, et al, Metabolism 2001, 50:407 413; Hauner, et al, J Clin Invest 1989, 84:1663 1670; Rodbell, et al, J Biol Chem 1966, 241:130 139).

ADSC can be isolated according to the following non-limiting method. First, isolated adipose tissue (i.e., fat tissue or liposuction fat) is rinsed with PBS containing 1% penicillin and streptomycin, minced into small pieces, then mixed with a solution containing 0.075% collagenase Type IA (Sigma-Aldrich, St. Louis, Mo.) at 5:1 v/v ratio of collagenase solution: adipose. After incubation for 1 hour at 37° C. with vigorous shaking, the product is then centrifuged at 220×g for 10 minutes at room temperature. Three layers are formed: the upper lipid layer, the middle collagenase layer and the bottom cellular pellet. The middle layer is collected and filtered through a 200 μm filter followed by centrifugation. The recycled collagenase Type IA, in the flow-thru, is used in a second round to digest the fresh adipose tissue again, using a higher ratio (7:1, by volume) than the first round. The bottom cellular pellet contains the stem cells.

ADSC or adipose tissue comprising ADSC can be preserved or stored prior to further purification, differentiation, administration to a subject, or any other use. Although it has been reported that the adipose tissue could be stored at room temperature for 24 hr and at 4° C. for 1-3 days, the viable cells in the adipose tissue decline dramatically in storage. The present application provides a method for preserving the viability of ADSC or adipose tissue which utilizes an adipose tissue preservation solution (ATPS), wherein the ATPS contains as its essential ingredient the enzyme superoxide dismutase (SOD). The superoxide dismutase can be isolated from human erythrocytes. In some embodiments, the ATPS consists of 200 mg/ml KH₂PO₄, 200 mg/L KCl, 2.16 g/L Na2HPO4.7H2O, 8 g/L NaCl, 30,000 units/L SOD, and 5 g/L Human serum albumin (HSA). To preserve adipose-derived stem cells, 1×10⁶ human adipose derived stem cells can be mixed with 1 ml of ATPS and stored at 4° C. One skilled in the art will recognize that the concentrations of reagents comprised by the ATPS may be altered to modest degrees without substantially affecting the desirable properties of the ATPS.

In most of the standard culture media for ADSC, animal serum is an essential component. However, integration of animal protein into the stem cells has been reported and is a major concern in human cell therapy. The present invention therefore provides methods and compositions for culturing ADSC in a lysate obtained from human blood platelets. Specifically, a medium with or without 10% FBS and 1-10% platelet lysate, e.g., 3-5% platelet lysate, can be used. The platelet lysate in the medium may be obtained by freezing and thawing platelets, then centrifuging the cellular debris. More detail is provided in the Examples herein.

The stromal vascular fraction derived from adipose tissue digestion consists of many type of cells, such as stem cells, endothelium, smooth muscle cell, and other terminally differentiated cells. Panning, an immuno-selection method, can be used to enrich a specific cell population from a diverse mixture of cell types. This method is based on the selective capabilities of antibodies bound to cell culture dishes. A mixture of cell types is cultured on the antibody-coated plates and allowed to bind for a short period of time. The non-adherent cells (those that do not bind antibody) can then be gently eluted from the culture dish allowing bound cells to be harvested. This method facilitated the development of other new technologies such as density-gradient separations and methods that exploit unique surface binding properties of specific cell types. For example, the development of magnetic bead technology allowed repetitive washing of bead-bound cells, greatly improving the potential purity of separations. The size and composition of the paramagnetic beads utilized by various companies varies significantly and further refinements and improvements have been regularly forthcoming.

Human adipose-derived stem cells can be selected using a combination of antibodies that, together, can be used to detect the presence of the CD34, CD90 and SSEA1 cell markers. Other antibodies can be used in addition to these markers, e.g., those markers disclosed in the published U.S. Pat. App. No. 20060147430 to Sayre et al. Following identification of positive cells, the cells can be cultured, studied further, or administered to subjects in one or more of the methods of treatment disclosed herein.

### III. Methods of Treatment and Administration of ADSC

ADSC may be administered to treat a number of mammalian pathologies, including erectile dysfunction, diabetes, tissue regeneration, wound healing, scarring, soft tissue defect, impotence, overactive bladder, incontinence and hair loss. ADSC may also be used for a variety of cosmetic purposes, including the treatment of wrinkles and breast and penile enhancement.

Adipose-derived stem cells or differentiated cells may be transplanted into the recipient where the cells will proliferate and differentiate to form new cells and tissues, thereby providing the physiological processes normally provided by that tissue. The term “transplanted” as used herein refers to transferring cells alone or cells that are embedded in a support matrix. The cells can be autologous or allogeneic. As used herein, the term “tissue” refers to an aggregation of similarly specialized cells united in the performance of a particular function. Tissue is intended to encompass all types of biological tissue including both hard and soft tissue. Soft tissue refers to tissues that connect, support, or surround other structures and organs of the body. Soft tissue includes muscles, tendons (bands of fiber that connect muscles to bones), fibrous tissues, fat, blood vessels, nerves, and synovial tissues (tissues around joints). Hard tissue includes connective tissue (e.g., hard forms such as osseous tissue or bone) as well as other muscular or skeletal tissue.

ADSC can be administered with a pharmaceutically acceptable carrier or excipients. The pharmaceutically acceptable excipients described herein, for example, vehicles, adjuvants, carriers or diluents, are well-known to those who are skilled in the art and are readily available to the public. Generally, the pharmaceutically acceptable carrier or excipient is one which is chemically inert to the therapeutic composition and one which has no detrimental side effects or toxicity under the conditions of use.

The choice of excipient or carrier will be determined in part by the particular therapeutic composition, as well as by the particular method used to administer the composition. Accordingly, there are a wide variety of suitable formulations of the pharmaceutical composition of the present invention. The formulations described herein are merely exemplary and are in no way limiting.

Often the physiologically acceptable carrier is an aqueous pH buffered solution. Examples of physiologically acceptable carriers include, but are not limited to, saline, solvents, dispersion media, cell culture media, aqueous buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

### IV. Compositions

The present invention further provides therapeutic compositions useful in practicing the therapeutic methods of this invention. A subject therapeutic composition includes, in admixture, a pharmaceutically acceptable excipient (carrier) or media and the ADSC of the present invention, including cells or tissues derived therefrom, alone or in combination with one or more bioactive agents, and at a strength effective for administration by various means to a patient experiencing cellular or tissue loss or deficiency.

The present invention provides therapeutic compositions for use in methods which comprise or are based upon ADSCs, including lineage-uncommitted populations of cells, lineage-committed populations of cells or tissues derived therefrom, along with a pharmaceutically acceptable carrier or media. Also contemplated are therapeutic compositions comprising bioactive agents that act on or modulate the ADSC of the present invention and/or the cells or tissues derived therefrom, along with a pharmaceutically acceptable carrier or media.

The preparation of cellular or tissue-based therapeutic compositions is well understood in the art. Such compositions can be formulated in a pharmaceutically acceptable media. The cells can be in solution or embedded in a matrix. The preparation of therapeutic compositions with bioactive agents (such as, for example, growth factors) as active ingredients is well understood in the art. The active therapeutic ingredient is often mixed with excipients or media which are pharmaceutically acceptable and compatible with the active ingredient. In addition, if desired, the composition can contain minor amounts of auxiliary substances such as wetting or emulsifying agents, pH buffering agents which enhance the effectiveness of the active ingredient.

A bioactive agent can be formulated into the therapeutic composition as neutralized pharmaceutically acceptable salt forms. Pharmaceutically acceptable salts include the acid addition salts (formed with the free amino groups of the polypeptide or antibody molecule) and which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The therapeutic compositions of the present invention are administered in a manner compatible with the dosage formulation, and in a therapeutically effective amount. The quantity to be administered depends, for instance, on the subject and debilitation to be treated, capacity of the subject's organ, cellular and immune system to accommodate the therapeutic composition, and the nature of the cell or tissue therapy, etc. Precise amounts of therapeutic composition required to be administered depend on the judgment of the practitioner and are peculiar to each individual. However, suitable dosages of the therapeutic composition of the present invention may range from about 0.05-100.0×10⁶ adipose-derived stem cells/10 mm of treatment site, per treatment site per day, preferably about 0.10-50.0×10⁶ adipose-derived stem cells/10 mm of treatment site, per treatment site per day, and more preferably about 0.5-5.0×10⁶ adipose-derived stem cells/10 mm of treatment site, per treatment site per day, and depend on the route of administration and the size of the treatment site. Suitable regimines for initial administration and follow on administration are also variable, but can include an initial administration followed by repeated doses at one or more intervals as desired or indicated (e.g. hours, days, weeks, months, or years) by a subsequent injection or other administration.

One of skill in the art may readily determine the appropriate concentration of cells for a particular purpose. In a non-limiting example, approximately 0.5×10⁶ adipose-derived stem cells/10 mm of treatment site per treatment site per day, are intradermally injected adjacent to, or within, the treatment site. Precise administration schedules for the therapeutic composition depend on the judgment of the practitioner and the desired result and are therefore peculiar, to a certain extent, to each individual.

The ADSC or differentiated cells of the present invention can be administered by injection into a target site of a subject, preferably via a delivery device, such as a tube, e.g., catheter. In one embodiment, the tube additionally contains a needle and/or a syringe, through which the cells can be introduced into the subject at a desired location. Specific, non-limiting examples of administering cells to subjects may also include administration by subcutaneous injection, intramuscular injection, or intravenous injection. If administration is intravenous, an injectable liquid suspension of cells can be prepared and administered by a continuous drip or as a bolus.

Cells may also be inserted into a delivery device, e.g., a syringe, in different forms. For example, the cells can be suspended in a solution contained in such a delivery device. As used herein, the term “solution” includes a pharmaceutically acceptable carrier or diluent in which the cells of the invention remain viable. The use of such carriers and diluents is well known in the art. The solution is preferably sterile and fluid to the extent that easy delivery via syringe exists. The solution is typically stable under the conditions of manufacture and storage and preserved against the contaminating action of microorganisms such as bacteria and fungi through the use of, for example, parabens, chlorobutanol, phenol, ascorbic acid, thimerosal, and the like. Solutions of the invention can be prepared by incorporating ADSC or differentiated cells as described herein, in a pharmaceutically acceptable carrier or diluent and, as required, other ingredients enumerated above, followed by filter sterilization.

The cells may be administered systemically (for example intravascularly, such as via artery or vein) or locally (for example directly into a myocardial defect under echocardiogram guidance, or by direct application under visualization during surgery). For such injections, the cells may be in an injectable liquid suspension preparation or in a biocompatible medium which is injectable in liquid state but becomes semi-solid at the site of damaged tissue. A conventional intra-cardiac syringe or a controllable endoscopic delivery device can be used so long as the needle lumen or bore is of sufficient diameter (e.g. 30 gauge or larger) that shear forces will not damage the cells being delivered.

Cells may be administered in any manner that permits them to graft to the intended tissue site and reconstitute or regenerate the functionally deficient area.

Support matrices into which the ADSC can be incorporated or embedded include matrices which are biocompatible, recipient-compatible and which degrade into products which are not harmful to the recipient. These matrices provide support and protection for ADSC and differentiated cells in vivo.

Natural and/or synthetic biodegradable matrices are examples of such matrices. Natural biodegradable matrices include plasma clots, e.g., derived from a mammal, collagen, fibronectin, and laminin matrices. Suitable synthetic material for a cell transplantation matrix must be biocompatible to preclude migration and immunological complications; and should be able to support extensive cell growth and differentiated cell function. It must also be degradable, allowing for a completely natural tissue replacement. The matrix should be configurable into a variety of shapes and should have sufficient strength to prevent collapse upon implantation. A variety of studies indicate that the biodegradable polyester polymers made of polyglycolic acid fulfill all of these criteria, as described by Vacanti et al., J. Ped. Surg., 23:3-9 (1988); Cima, et al, Biotechnol. Bioeng. 38:145 (1991); Vacanti, et al., Plast. Reconstr. Surg., 88:753-9 (1991). Other synthetic biodegradable support matrices include synthetic polymers such as polyanhydrides, polyorthoesters, and polylactic acid. Further examples of synthetic polymers and methods of incorporating or embedding cells into these matrices are also known in the art. See e.g., U.S. Pat. Nos. 4,298,002 and 5,308,701.

Attachment of the cells to the polymer may be enhanced by coating the polymers with compounds such as basement membrane components, agar, agarose, gelatin, gum arabic, collagens types I, II, III, IV and V, fibronectin, laminin, glycosaminoglycans, mixtures thereof, and other materials known to those skilled in the art of cell culture. All polymers for use in the matrix must meet the mechanical and biochemical parameters necessary to provide adequate support for the cells with subsequent growth and proliferation.

One of the advantages of a biodegradable polymeric matrix is that angiogenic and other bioactive compounds can be incorporated directly into the support matrix so that they are slowly released as the support matrix degrades in vivo. As the cell-polymer structure is vascularized and the structure degrades, adipose tissue derived stem cells may differentiate according to their inherent characteristics. Factors, including nutrients, growth factors, inducers of differentiation or de-differentiation (i.e., causing differentiated cells to lose characteristics of differentiation and acquire characteristics such as proliferation and more general function), products of secretion, immunomodulators, inhibitors of inflammation, regression factors, bioactive agents which enhance or allow ingrowth of the lymphatic network or nerve fibers, hyaluronic acid, and drugs, which are known to those skilled in the art and commercially available with instructions as to what constitutes an effective amount, from suppliers such as Collaborative Research, Sigma Chemical Co., vascular growth factors such as vascular endothelial growth factor (VEGF), epidermal growth factor (EGF), and heparin binding epidermal growth factor like growth factor (HB-EGF), could be incorporated into the matrix or provided in conjunction with the matrix. Similarly, polymers containing peptides such as the attachment peptide RGD (Arg-Gly-Asp) can be synthesized for use in forming matrices (see, e.g., U.S. Pat. Nos. 4,988,621, 4,792,525, 5,965,997, 4,879,237 and 4,789,734).

In another example, the cells may be transplanted in a bioabsorbable gel matrix (such as Gelfoam from Upjohn Company), which polymerizes to form a substrate in which the adipose tissue derived stem cells or differentiated cells can grow. A variety of encapsulation technologies have been developed (e.g. Lacy et al., Science 254:1782-84 (1991); Sullivan et al., Science 252:718-712 (1991); WO 91/10470; WO 91/10425; U.S. Pat. No. 5,837,234; U.S. Pat. No. 5,011,472; U.S. Pat. No. 4,892,538).

PLGA or poly(lactic-co-glycolic acid) is a Food and Drug Administration (FDA)-approved copolymer which is used in a host of therapeutic devices, owing to its biodegradability and biocompatibility. PLGA is synthesized by means of random ring-opening co-polymerization of two different monomers, the cyclic dimers (1,4-dioxane-2,5-diones) of glycolic acid and lactic acid. Common catalysts used in the preparation of this polymer include tin(II) 2-ethylhexanoate, tin(II) alkoxides, or aluminum isopropoxide. During polymerization, successive monomeric units (of glycolic or lactic acid) are linked together in PLGA by ester linkages, thus yielding a linear, aliphatic polyester as a product. PLGA has been successfully used as a biodegradable polymer because it undergoes hydrolysis in the body to produce the original monomers, lactic acid and glycolic acid. These two monomers are by-products of various metabolic pathways in the body. Since the body is able to effectively break down the two monomers, there is no systemic toxicity associated with using PLGA for drug delivery or biomaterial applications. As shown in the Examples presented herein, ADSCs mixed with PLGA/carboxymethylcellulose (CMC) have a greater tendency to remain at the injected area compared to ADSCs mixed with saline.

### V. Genetically Modified Cells

ADSC can be used without modification as the cells can differentiate along multiple lineages. In some applications, it is preferable to use cells without genetic or chemical modification.

In addition, however, ADSCs can be engineered to contain genes that express select wound healing proteins, enzymes or drugs, for expression and delivery at the target site, to augment wound healing. For example, the ADSCs could be engineered to express beneficial genes. Such genes include PDX1, VP16, FGF2, VEGF, BDNF, IGF, TGF, NGF and other neurotrophic and vasculotrophic growth factors. Injection of a specifically engineered ADSC may help the regeneration of certain tissues; for example, BDNF for nerves.

In some embodiments, the ADSC expressing at least one genotypic or phenotypic characteristic of a chondrocyte is genetically modified to express exogenous genes or to repress the expression of endogenous genes and implanted into an animal. The invention provides a method of genetically modifying such cells and populations prior to implantation.

The desired gene is generally included in a recombinant expression cassette that also includes regulatory elements, e.g., a promoter and/or enhancer, to drive expression. In some embodiments, the promoter is a constitutive promoter. In some embodiments, the promoter is inducible. Recombinant vectors with various types of promoters are commercially available and familiar to those of skill in the art.

A nucleic acid construct comprising a promoter and the sequence of interest can be introduced into a recipient cell either as a non-replicating DNA (or RNA) molecule, which can either be a linear molecule or, more preferably, a closed covalent circular molecule. Since such molecules are incapable of autonomous replication without an origin of replication, the expression of the gene can occur through the transient expression of the introduced-sequence. Alternatively, permanent expression can occur through the integration of the introduced DNA sequence into the host chromosome.

In some embodiments, a vector is employed which is capable of integrating the desired gene sequences into the host cell chromosome. Cells which have stably integrated the introduced DNA into their chromosomes can be selected by also introducing one or more markers which allow for selection of host cells which contain the desired nucleic acid sequence. The marker, if desired, can provide for prototrophy to an auxotrophic host, biocide resistance, e.g., resistance to antibiotics, or heavy metals, such as copper, or the like. The selectable marker gene sequence can either be directly linked to the DNA gene sequences to be expressed, or introduced into the same cell by co-transfection. Preferably, expression of the marker can be quantified.

In some embodiments, the introduced nucleic acid molecule can be incorporated into a plasmid or viral vector capable of autonomous replication in the recipient host. Any of a wide variety of vectors can be employed for this purpose. Factors of importance in selecting a particular plasmid or viral vector include: 1) the ease with which recipient cells that contain the vector can be recognized and selected from those recipient cells which do not contain the vector; 2) the number of copies of the vector which are desired in a particular host; and 3) whether it is desirable to be able to “shuttle” the vector between host cells of different species.

Eukaryotic vectors include for example, vaccinia virus, SV40, retroviruses, adenoviruses, adeno-associated viruses and a variety of commercially available, plasmid-based mammalian expression vectors that are familiar to those experienced in the art.

Once the vector or nucleic acid molecule containing the construct(s) has been prepared for expression, the DNA construct(s) can be introduced into an appropriate host cell by any of a variety of suitable means, i.e., transformation, transfection, viral infection, conjugation, protoplast fusion, electroporation, particle gun technology, calcium phosphate-precipitation, direct microinjection, and the like. After the introduction of the vector, recipient cells are grown in a selective medium, which selects for the growth of vector-containing cells. Expression of the cloned gene molecule(s) results in the production of the heterologous protein.

Introduced DNA being “maintained” in cells should be understood as the introduced DNA continuing to be present in essentially all of the cells in question as they continue to grow and proliferate. That is, the introduced DNA is not diluted out of the majority of the cells over multiple rounds of cell division. Rather, it replicates during cell proliferation and at least one copy of the introduced DNA remains in almost every daughter cell. Introduced DNA can be integrated directly into the cell's genome or exist as an extrachromosomal element, or episome. In order for an episome not to be diluted out during cell proliferation, a selectable marker gene can be included in the introduced DNA and the cells grown under conditions where expression of the marker gene is required. Even in the case where the introduced DNA has integrated in the genome, a selectable marker gene may be included to prevent excision of the DNA from the chromosome.

The genetically altered cells can then be introduced into the subject by a variety of methods under conditions for the transgene to be expressed in vivo. As a non-limiting example, the transgene can encode for the production of PDX1, VP16, or an extracellular matrix protein, such as collagen. The cells containing the transgene for the extracellular matrix protein can then be introduced into the animal. Alternatively, the cells containing the transgene are injected intraperitoneally or into some other suitable organ depot site.

### VI. Therapeutic Applications

A. ADSC Treatment of Overactive Bladder, Urinary Tract Injury, and Other Incontinence Disorders

Overactive bladder (OAB) is a health problem characterized by the sudden and compelling desire to pass urine. OAB affects one sixth of adults aged 18 years and over in the US and Europe (33 million people in the US). Its prevalence is even higher in older populations, reaching 41% and 31% respectively for men and women over 75 years of age. In diabetic patients, OAB is also one of the most significant clinical manifestations in the early stages of disease occurring in 55% of diabetic patients. In later stages, some patients develop decreased bladder sensation, bladder distension and eventually overflow incontinence. Thus, as the developed and the developing countries are experiencing an accelerated aging population and an increasingly obese society (obesity is a significant risk factor for diabetes), OAB is undoubtedly one of the most important health issues facing developing countries.

At present, the only FDA-approved treatment for OAB is a class of drugs that are antagonistic against the muscarinic receptors. These drugs are tolterodine, oxybutynin, trospium, solifenacin, and darifenacin. However, because the muscarinic receptors exist in at least 5 different isoforms that are distributed throughout the body, these anticholinergic drugs often produce many adverse effects such as blurred vision, dry mouth, urinary retention, constipation, and central nervous system (CNS) effects such as somnolence and confusion. Moreover, the medications are not curative and need to be taken for the patient's rest of life.

In some embodiments, the invention provides a method for treating OAB, urinary tract injury, or incontinence by injecting autologous (or syngeneic) ADSCs into the bladder, urethra, or site of injury of an individual. Because ADSC injection is a localized treatment it is not expected to have systemic effects. Moreover, the injected ADSC can integrate into the host tissue (bladder and/or urethra), thereby offering long-term therapeutic benefits. Thus, the ADSC treatment can address the cause of the incontinence or OAB. If used before the development of end-stage conditions such as overflow incontinence this therapy can serve as an excellent preventive measure.

B. ADSC Treatment of Impotence

Impotence (or erectile dysfunction (ED)) is a common problem globally. In the United States alone, approximately 20 million men suffer from ED. Penile erection is a neurovascular event modulated by psychological and hormonal status. Upon sexual stimulation, the nerve impulses release neurotransmitters (nitric oxide is the most important one) from the cavernous nerve terminals and relaxing factors from the endothelial cells. This release results in a relaxation of arterial and arteriolar smooth muscles, a drop in peripheral resistance, and a several-fold increase in arterial flow. In the meantime, relaxation of the trabecular smooth muscle increases compliance of the sinusoids, facilitating expansion of the entire sinusoidal system against the tunica albuginea and resulting in decreased venous flow.

The penis is richly innervated by autonomic (sympathetic and parasympathetic) and somatic (sensory and motor) nerves. The somatic nerves are primarily responsible for penile sensation and the contraction and relaxation of the extracorporeal striated muscles. The autonomic nerves (sympathetic and parasympathetic) innervate the corpora cavernosa and corpus spongiosum to control the hemodynamics during erection and detumescence. The cavernous nerves are the terminal branches of the autonomic nerve that innervate the penis. These fine nerve fibers are only millimeters from the prostate and are easily damaged during radical excision of the rectum, bladder and prostate.

Many studies have shown a significant association of ED and urological surgeries and radiation (cystoprostatectomy, transurethral prostate surgery, radical prostatectomy, and cryoablation of the prostate). The incidence of ED was reported as 50-70% after nerve-sparing radical prostatectomy and close to 90% after cryoablation of the prostate. Since its introduction in 1998, a PDE5 inhibitor, sildenafil (Viagra), has been quite successful in treating ED of various causes except neurogenic ED after radical pelvic surgeries. So far, PDE5 inhibitors have not been shown to help regeneration of the cavernous nerves. ED is also a very common (up to 50%) among men with diabetes mellitus. PDE5 inhibitors also result in poor response rates in diabetic men (about 45% successful).

In some embodiments, ADSCs are administered locally to the corpus cavernosum of a subject suffering from impotence as a result of pelvic surgeries such as surgery for cancer of the prostate, bladder and rectum. The ADSCs can facilitate nerve recovery and thus improve or cure the impotence from injury to the cavernous nerve.

In some embodiments, ADSCs are administered locally to the corpus cavernosum of a subject suffering from impotence associated with diabetes. Because ADSCs can induce new blood vessels formation and improve blood circulation, they can also be administered locally to the corpus cavernosum of a subject suffering from vasculogenic impotence associated with high blood pressure, elevated cholesterol/lipid, arteriosclerosis/atherosclerosis or pelvic/perineal injury.

C. ADSC Treatment for Healing Wounds

ADSCs can also be administered to subjects for the purpose of accelerating wound healing and reducing scar formation. This has application both for improved cosmesis (i.e. to improve scar healing purely for cosmetic purposes), and, as an adjunct to improve surgical scars within the body (e.g. excessive scarring at surgical anastomosis sites can lead to surgical complications, such as anastomosis site contractures. Local injection of ADSC at the time of surgery, or, in a delayed fashion, can improve surgical tissue site healing and reduce the incidence of post-surgical complications due to excessive scarring.)

Accordingly, in some embodiments, ADSCs are injected subcutaneously at the desired location, e.g., along the suture site of a surgical wound. In certain embodiments, the use of support material such as Matrigel or microspheres with CMC may be introduced to further localize the ADSCs to the site of treatment. As described in more detail in the Examples, the introduction of ADSCs to the wound site leads to the differentiation of the ADSCs in a manner that mimics the body's natural response to a wound. For example, ADSCs appear to spontaneously differentiate into the other cell types that comprise the natural wound healing apparatus, e.g., fibroblasts and inflammatory cells or newly formed blood vessels.

In some embodiments, suture material (absorbable or permanent) or permanently implanted prosthetic devices can be coated with scaffolds onto which ADSC are seeded prior to implantation of the suture material or prosthetic device into the body. In this way, ADSC are not only delivered to the surgical site, but serve as an interface with the body during tissue healing.

D. ADSC Treatment for Diabetes

In some embodiments, the invention provides a method for treating diabetes by administering to a subject an ADSC that has been transfected with an appropriate vector and transformed into an insulin producing cell. As described in more detail in the Examples, transfection of ADSCs with lentivirus expressing PDX1 or VP16 (or any other gene known to stimulate the transformation of stem cells into insulin producing islet cells) leads to the expression of the PDX or VP16, and the transformation of the cells into insulin producing islet cells, or insulin producing ADSC (IPADSC). Alternative methods can be used to drive expression of PDX 1 or VP 16, such as introduction of a promoter element to drive expression of the endogenous gene, or introduction of an expression vector by other means, such as liposomal means. Transformation of isolated cells will be appreciated as routine to those of skill in the art.

After transformation, IPADSC can be administered to an individual in need thereof. Such individuals include those at risk of diabetes (e.g., with a family history of diabetes, with high fasting blood glucose, or overweight individuals), as well as those diagnosed with either type 1 or type 2 diabetes. The IPADSC can be injected into the portal vein of a diabetic subject, as in human islet cell transplantation (see, e.g., Shapiro et al. (2006) NEJM, 355:1318-1330).

For individuals with type 1 diabetes, unmodified ADSC or IPADSC can be introduced to alleviate insulin deficiency. For type 2 diabetes, the treatment can be adjusted depending on the stage of the disease. During early stage type 2 diabetes, there is insulin resistance, and hyperinsulinemia, whereas in later stages, there is hypoinsulinemia. Unmodified ADSC can be used to improve insulin resistance and thus prevent or reverse many harmful effects associated with type 2 diabetes.

In some embodiments, unmodified ADSC can be injected (e.g., intraportally, intraperitoneally, or subcutaneously). Our experience has shown that the ADSC mitigate the effects of type 2 diabetes, such as erectile dysfunction, peripheral neuropathy, and poor blood circulation. In addition, ADSC administration results in decreased serum blood glucose levels in animals with type 2 diabetes, suggesting that ADSC can improve insulin resistance observed in type 2 diabetics.

E. ADSC Treatment of Peripheral Neuropathy

Peripheral neuropathy is characterized by decreased sensation. Decreased peripheral sensation leads to increased peripheral injuries, as the subject cannot feel when he/she has injured the extremity. They are less apt to withdraw the limb from pain, and/or, they are less apt to discover the injury, owing to lack of complete sensation. Injuries become chronic, and ultimately infected. The additional effect of poor wound healing, from diabetes, accelerates the path toward uncontrolled infection at the injury site. Ultimately, many diabetics require amputation of injured extremities.

The condition can arise because of nerve injury, or because of systemic disorders, such as diabetes, hormone imbalance, kidney disease, autoimmune disorders, alcoholism and vitamin deficiencies. Regardless of the cause, ADSC can be used to ameliorate the condition and restore sensation to the affected tissue. In some embodiments, ADSC are administered locally, e.g., to a site of reduced sensation. In some embodiments, ADSC are administered systemically, and the cells populate the damaged sites. In some embodiments, the ADSC injected systemically or to a different site produce cytokines and growth factors and improve insulin resistance to allow natural repair of the nerves and nerve-related microvasculature damaged by diabetes.

F. ADSC Treatment for Other Conditions

In related embodiments, the invention provides methods of treating a variety of other conditions using ADSCs. The methods include treatments for restoring hair growth in a subject by injecting ADSCs into the area of the subject's skin where hair follicle structures have been damaged, e.g., by chemotherapy (chemotherapy induced alopecia) or other non-specific damage to vital hair follicle structures, including radiation damage, skin injuries, and aging. Similarly, ADSCs can be used to replenish natural hair follicle “stem cell” reservoirs residing in the “hair bulge” component of the hair follicle. An alternative mechanism of ADSCs is to provide new blood vessel/improve circulation to the damaged hair follicle to facilitate its recovery and hair growth.

Similarly, wrinkles or soft tissue defect may be treated in a subject by local subcutaneous injection of ADSCs at the site of the wrinkling or soft tissue defect. ADSCs can be also be injected to cosmetically enhance the appearance of the penis of a subject, (e.g., to increase its circumference, to enhance the appearance of scarred or disfigured tissue, or to replace tissue lost due to trauma, in an accident. Similarly, ADSC can be used as a cosmetic adjunct in breast enhancement and tissue “bulking” procedures (e.g. “butt, hip or vaginal augmentation). To achieve the desired size/shape, ADSCs can be grown on a scafford made of PLGA and then implanted to the tissue/organ. ADSCs can also be used to replenish gonadal cells, e.g., Leydig cells, Sertoli cells, and spermatogonia-derived cells, in a subject in need thereof.

The present invention now is described more fully by the following Examples. This invention may, however, be embodied in many different forms and should not be construed as limited to the embodiments set forth herein; rather, these embodiments are provided so that this disclosure is thorough and complete, and fully conveys the scope of the invention to those skilled in the art.

### Examples

### I. Method for Isolating ADSC Using Recycled Collagenase

Isolated adipose tissue (i.e., fat tissue or liposuction fat) is rinsed with PBS containing 1% penicillin and streptomycin, minced into small pieces, then mixed with a solution containing 0.075% collagenase Type IA (Sigma-Aldrich, St. Louis, Mo.) at 5:1 v/v ratio of collagenase solution: adipose. After incubation for 1 hour at 37° C. with vigorous shaking, the product is then centrifuged at 220×g for 10 minutes at room temperature. Three layers are formed: the upper lipid layer, the middle collagenase layer and the bottom cellular pellet. The middle layer is collected and filtered through a 200 μm filter followed by centrifugation. The recycled collagenase Type IA in the flow-thru, is used to digest fresh adipose tissue (and thus decrease the cost of purchasing large amount of collagenase), using a higher ratio (7:1, by volume) than the first round.

II. Preserving ADSC with Superoxide Dismutase

This Example describes a new adipose tissue preservation solution (ATPS) containing superoxide dismutase isolated from human erythrocytes to preserve cell viability. The ATPS consists of 200 mg/L KH₂PO₄, 200 mg/L KCl, 2.16 g/L Na2HPO4.7H2O, 8 g/L NaCl, 30,000 units/L Superoxide Dismutase (SOD), and 5 g/L Human serum albumin (HSA).

The ATPS was used to preserve the human adipose tissue at 4° C. for 24 and 48 hr. The preserved adipose tissues were used to isolate adipose derived stem cells according to the procedures described herein. The yield of adipose-derived stem cells obtained from the ATPS-preserved issue and freshly harvested adipose tissue from two patients was compared. The yield of ADSC was 85% at 24 hours and 65% at 48 hours as compared to freshly harvested adipose tissue. This is much better than the yields obtained without ATPS as reported by Matsumoto et al., Plast. Reconstr. Surg. (2007) 120(6):1510-7.

The ATPS has also been tested for preservation of adipose stem cells. 1×10⁶ human adipose derived stem cells were mixed with 1 ml of ATPS and stored at 4° C. After 48 hours, a cell viability test (the tryptan blue method) was performed. The results indicated that less than 5% of cells were damaged after 48 hours of preservation with ATPS.

### III. Method for Isolating a Subpopulation of Multipotent Population of ADSC

[***Please confirm that the population here is “multipotent.” Is there a better description for CD34+/CD90+/SSEA1+?]

ADSC are multipotent. A combination of cell-surface antigens can be used to isolate a subpopulation of multipotent ADSC, though for many applications discussed herein, ADSC can be used without such separation. Efficient and accurate separation of specific cell types can be an aspect of many research projects. Antibodies can be used for cell separation, e.g., using panning or magnetic bead technology.

The stromal vascular fraction derived from adipose tissue digestion consists of many type of cells, such as stem cells, endothelium, smooth muscle cell, and other terminally differentiated cells. The magnetic cell system provides excellent sorting of magnetic-bead-labeled cells.

Freshly isolated ADSC were analyzed by flow cytometry for cell surface antigen expression according to the manufacturer's protocol. The cells were incubated with primary antibody (Table 1) in 50 μl wash buffer (PBS containing 1% FBS and 0.1% Na₃N) for 30 minutes on ice, followed by another incubation with FITC-conjugated secondary antibody (goat anti-IgG). The cells were then rinsed twice with wash buffer, fixed with 1% para-formaldehyde in PBS, and analyzed by a fluorescence-activated cell sorter (FACS Vantage SE; Becton Dickinson). The results were analyzed with FlowJo software (Tree Star, Inc., Ashland, Oreg.). Cell antigens analyzed were CD13, CD31, CD34, CD90, CD105, CD133, SSEA-1 (stage specific embryonic antigen 1), and telomerase. The expression levels are presented in Table 2.

TABLE 1
Antibodies used in this study

| Target protein | Supplier |
|---|---|
| CD13 | Santa Cruz Biotech, Santa Cruz, CA |
| CD31 | Santa Cruz Biotech, Santa Cruz, CA |
| CD34 | Santa Cruz Biotech, Santa Cruz, CA |
| CD90 | Santa Cruz Biotech, Santa Cruz, CA |
| CD105 | Chemicon, Temecula, CA |
| CD133 | Abcam Inc, Vambridge. MA |
| SSEA1 | Abcam Inc, Vambridge. MA |
| Telomerase | Abcam Inc, Vambridge. MA |

TABLE 2
Expression level of cellular markers in adipose derived stem cells

| Cellular markers | Positive % (mean ± standard deviation) |
|---|---|
| CD13 | 6.5 ± 1.1 |
| CD31 | 24.1 ± 3.8 |
| CD34 | 67.9 ± 20 |
| CD90 | 87 ± 21.4 |
| CD105 | 56 ± 18.6 |
| CD133 | 6.9 ± 2.5 |
| SSEA1 | 25.7 ± 6.9 |
| Telomerase | 2.7 ± 0.98 |

Antibody Cocktail

According to the result of flow cytometry, three markers were selected as positive cellular markers to separate the adipose derived stem cells. The antibody cocktail include mouse anti-CD34, mouse anti-CD90 and mouse anti-SSEA1 in a ratio of 2:3:5.

Positive Selection of CD34(+)/CD90(+)/SSEA1(+) by MACS

Human adipose derived stem cells were incubated with CD34 (+)/CD90 (+)/SSEA1 (+). Positive cells were selected with Pan-mouse antibody magnetic beads.

Differences in Proliferation: CD34(+)/CD90(+)/SSEA1(+) versus CD34((−)/CD90(−)/SSEA1(−) Cells

The positive cells selected using the CD34(+)/CD90(+)/SSEA1(+) cocktail and the negative depleted cells were cultured and used for the cell proliferation assay by the MTT test). The result showed that the positive cell grow faster than the negative selected cell.

Difference in Cytokine Secretion: CD34 (+)/CD90(+)/SSEA1(+) versus CD34(−)/CD90(−)/SSEA1(−) Cells

The cell culture medium from positive and negative selected cells was used to check the production of cytokines by the cytokine arrays. The result indicated that positive selected cell secrete more cytokines, such as MCP1, b-NGF, TIMP-1 TNF-a, IL-1b, CINC-1 et al.

Difference in Recovering Erectile Function In Vivo: CD34(+)/CD90(+)/SSEA1(+) versus CD34(−)/CD90(−)/SSEA1(−) Cells

The positive and negative selected ADSCs were cultured and injected into rat corpus cavernosum after crush injury of the cavernous nerves. Four weeks later, erectile function was assessed by neurostimulation. The results showed that the positively selected cells significantly improved the erectile function while the negative cells did not.

### IV. Culturing ADSCs in Platelet Lysate

In most of the standard culture media for ADSCs, animal serum is an essential component. However, integration of animal protein into the stem cells has been reported and this is a major concern in human cell therapy. Here we describe a novel method for culturing ADSCs in a platelet lysate medium.

Platelet lysate was obtained from whole blood according to the following procedure. Whole blood was drawn into four 50 ml sterile plastic tubes containing sodium citrate dehydrate, and centrifuged at 350×g for 10 minutes. The platelet-rich plasma fraction was washed with an equal volume of Phosphate Buffer saline (PBS) containing 0.38 mg/ml of sodium citrate dihydrate. Platelets were then centrifuged at 510×g for 10 minutes and the pellet was suspended in DMEM to a final concentration of 1-2×10⁹ cells/ml. Platelet lysis, (and consequent release of chemotactic and growth factors) was obtained by a single cycle of freezing (80° C.) and thawing (37° C.). The platelet lysate in DMEM was centrifuged at high speed (12,000×g, 10 min.) to remove cell membranes, and the supernatant was extracted and stored at −80° C.

To test the ability to maintain and promote cell growth and proliferation, ADSCs were cultured in serum free medium, and in medium containing 10% FBS, 2% platelet lysate and 4% platelet lysate. The proliferative effect of platelet lysate on ADSC was evaluated by the MTT test. The result indicated that 4% platelet lysate has the same effect in supporting cell growth and proliferation as 10% FBS.

### V. Sixty Micron PLGA Microspheres and Carboxymethylcellulose as Carriers for ADSCs

An injectable poly(lactic-co-glycolic acid) (“PLGA”) solution was prepared by first mixing 60 μm PLGA microspheres with 0.5% carboxymethylcellulose (CMC), dissolved in PBS) to reach a 1×10⁷/ml microsphere suspension. Approximately 1-3×10⁶ ADSCs were mixed with 200-800 μl PLGA/CMC mixture. The ratio of ADSCs to PLGA microsphere is 1:10. After incubating on ice for 30 minutes, the ADSCs/PLGA mixture was injected into bladder and the corpus cavernosum of the penis. As a control, ADSCs mixed with saline were injected to a different group of animals. Four weeks later, the animals were killed and tissues and examined. The results showed that many more ADSCs were retained in the injected area in the ADSC/PLGA group than the saline group.

VI. Treating Wounds and other Conditions with ADSC

The degree to which ADSCs could spontaneously differentiate into blood vessels was assessed. For example, ADSCs were injected percutaneously into the subcutaneous space in healthy rats. Histological studies showed that the ADSCs differentiated into a wide variety of local cell types, including blood vessels, fat, muscle, connective tissue/fibroblasts, and peri-follicular (surrounding a skin hair follicle) cells.

ADSCs were also injected subcutaneously along the suture site of a surgical wound. One side of the suture line received subcutaneous injection of ADSC suspended in buffer, whereas the other side received only injection of buffer. Results showed that the side of the wound that received ADSC developed a significantly greater density of blood vessels. Furthermore, the degree of scar formation on the side of the wound that received ADSC appeared more attenuated as compared to the control side. Such experiments were repeated wherein the design was varied so that identical wounds were created in the same animal, and the experimental wound received ADSCs while the control wound, otherwise identical, received no ADSCs. The results were highly reproducible.

Additional studies showed that injected ADSCs preferentially differentiate into blood vessels within a wound environment. A reagent called Matrigel was used to introduce the ADSCs into the wound environment. Matrigel is a biocompatible but otherwise inert material that serves as a dense gel which allows diffusion of oxygen and micronutrients, but which is sufficiently dense to prevent local tissue or cell ingrowth. Importantly, it exists in a liquid state at artificially cold temperatures, and in a semi-solid state at body temperature.

The negative control was Matrigel without ADSC injected subcutaneously into the dorsal area of an adult Sprague-Dawley rat, and then resected after 10 days. The resected Matrigel mass was then processed, sectioned, and stained (using standard immunohistochemical techniques) for BrdU. There were no ADSC and the scant number of other cells present within the Matrigel. In the experimental group, ADSCs were suspended within a set volume of Matrigel, and then percutaneously injected into the subcutaneous space of a rat's dorsal hump. In the same rat, on the contralateral side of their dorsal hump, we injected an identical volume of Matrigel, without ADSC, as a control. The material was allowed to remain within the wound site for 10 days, and then excised from the wound space, sectioned, and histologically evaluated. Results showed that the labeled ADSC suspended in the Matrigel differentiated into blood vessels /endothelial cells. Furthermore, the edges of the Matrigel in apposition to the wound cavity demonstrated a greater density of blood vessels, as compared to the center. Again, the ADSC were labeled with a nuclear marker, which confirmed that the neovascularity visualized corresponded to ADSC, not local blood vessel ingrowth. The control injections of Matrigel alone demonstrated no blood vessel formation.

The results described above show that the host tissue environment within the target site influences the types of cells that result from the differentiation of ADSCs. Injected ADSCs respond to a wound environment by differentiating preferentially into blood vessels, which is consistent with the natural wound healing response, i.e., local tissue hypoxia influences ADSCs to differentiate preferentially into blood vessels. Furthermore, ADSCs appear to spontaneously differentiate into the other cell types that comprise the natural wound healing apparatus, e.g., fibroblasts and inflammatory cells. Specific wound applications include, without limitation, the prevention of scarring/stricture at surgical anastomoses, radiation-induced wounds, surgical stomas (e.g., colostomies, urostomoies), and cosmetic surgical wounds. ADSC treatment can also be used to prevent the development of pressure-sores (decubitus ulcers) or promote the healing of existing pressure-sores.

VII. Treating Wrinkles with ADSC

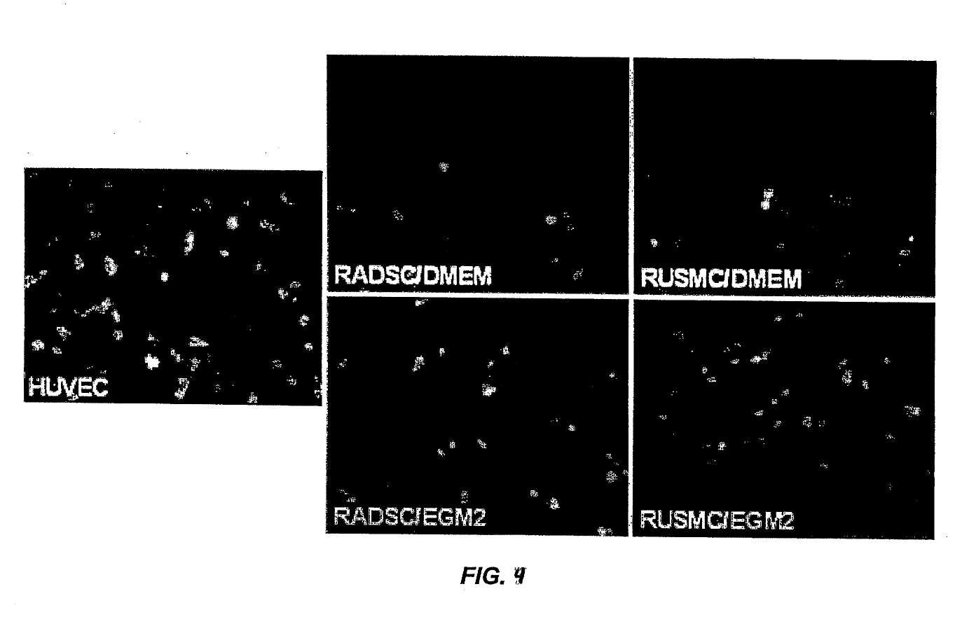

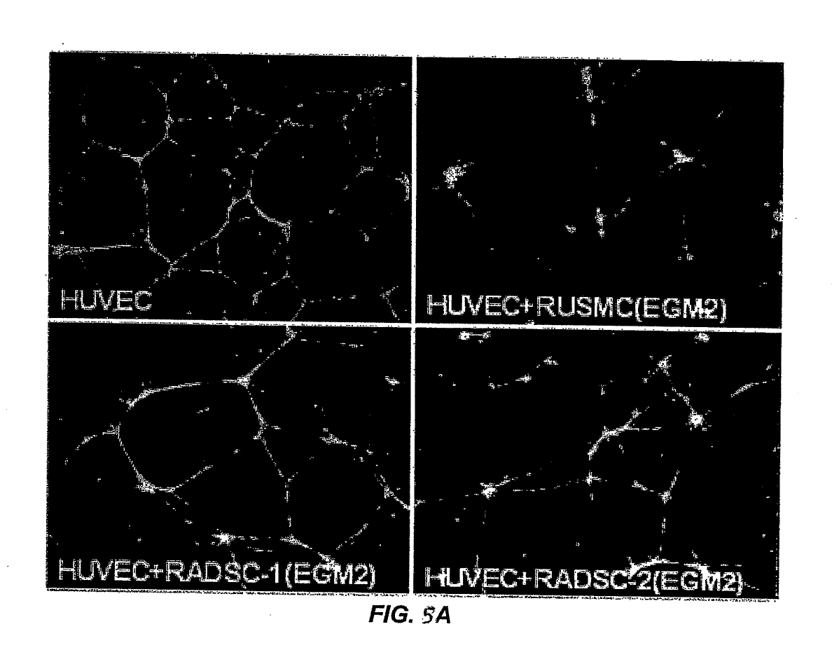

By injecting ADSCs to subcutaneous tissue, the tissues can be filled with the cells and more blood vessels will be formed because the ADSCs produce endothelial growth factor. FIG. 4 shows the results of experiments wherein rat subcutaneous tissues injected with stem cells were compared to those areas injected with saline. Where ADSC cells were injected, the cells stain positive for a marker (BrdU) and there are more small vessels in the area.

### VIII. Generation of Insulin Producing ADSC (IPADSC)

PDX1 is a key regulatory gene in beta cell (insulin producing cells) development and VP16 is an engineered version of PDX1 (see, e.g., Tang et al., Laboratory Investigation 86:829-841 (2006); Cao et al., Diabetes 53:3168-3178 (2004); Tang et al., Laboratory Investigation 86:83-93 (2006)). This Example describes results showing that, after transfection with Lentivirus carrying the PDX gene or VP16, ADSCs can be transformed into insulin-producing cells. These cells can be then injected to the portal vein (as in human islet cell transplantation) to help diabetic patients.

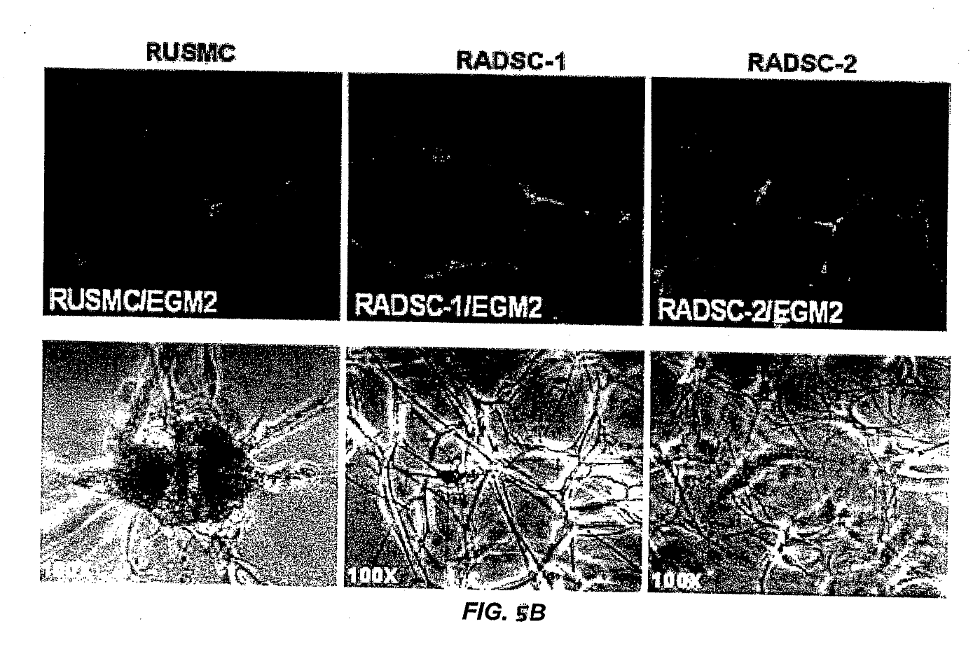

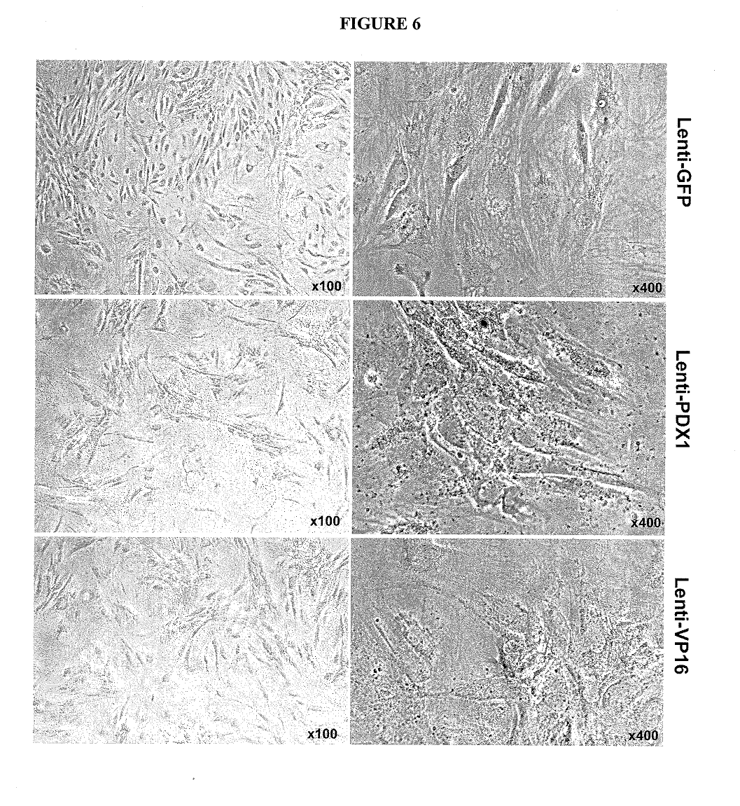

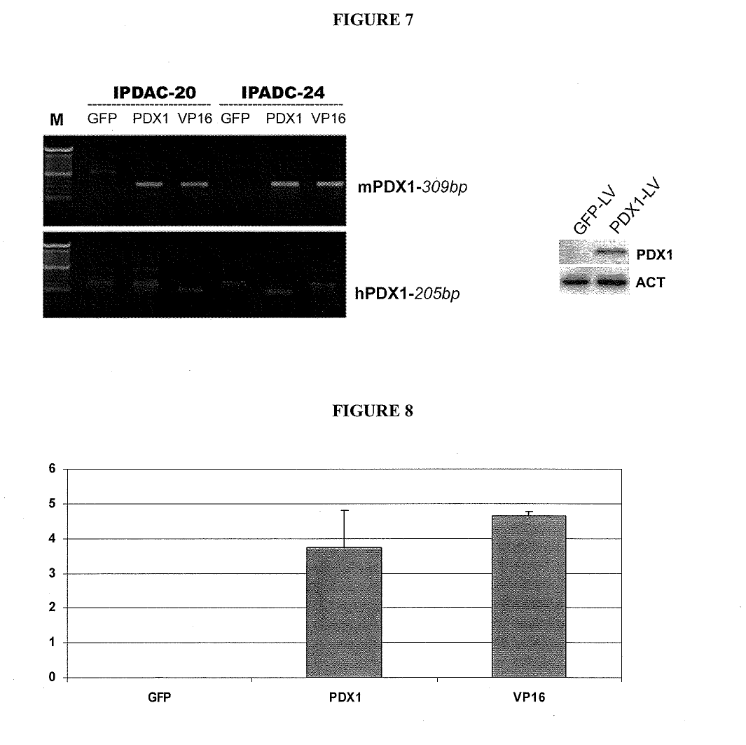

Human ADSCs (cell lines 20 and 24) were transfected with Lenti-PDX1, Lenti-VP16, or Lenti-GFP (GFP, green fluorescence protein), with the latter serving as a negative control. After transfection, the cells were cultured in differentiation medium. After 21 days, the morphology of GFP-transfected cells remained unchanged while that of PDX1- or VP 16-transfected cells changed significantly. In addition, the PDX1- or VP16-transfected cells appeared to be secreting granular materials, as shown in FIG. 6. PDX1 mRNA expression by the transfected cells was confirmed and measured by RT-PCR (FIG. 7). Western blots were used to confirm expression of the PDX1 protein (FIG. 7).

The production of insulin by transfected cells was also analyzed by staining cells with an anti-insulin antibody.

FIG. 8 shows the results of ELISA studies to quantify the levels of expression of insulin in ADSCs transfected with PDX1 or VP16. Both vectors lead to the production of similar amounts of insulin by the transfected ADSCs.

In another study we chose two human and five rat ADSC lines as candidates for transduction with Pdx1. Additionally, the two human ADSC lines were also transfected with Pdx1 -VP16 (PV16). Transduction with GFP served as a negative control as well as for the determination of transduction efficiency, which was found to be greater than 95% (percentage of cells displaying green fluorescence). Nuclear staining with DAPI (blue) was used to locate cells.

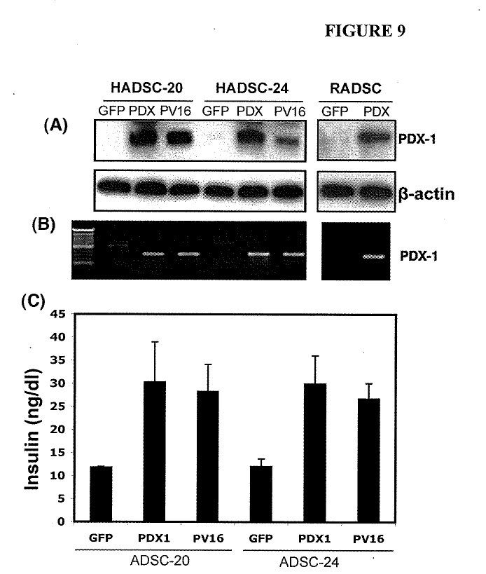

One week after transduction, the Pdx1- and PV16-transduced cells, but not the GFP-transduced cells, exhibited a morphology suggesting the secretion of insulin granules by phase contrast, which was subsequently confirmed by immunofluorescence staining. RT-PCR and Western blot analyses also confirmed Pdx1 expression in Pdx1- and PV16-transduced cells but not in GFP-transduced cells (FIG. 9). Finally, ELISA analysis showed the static production of insulin in Pdx1-transduced cells (FIG. 9); hence, insulin-producing ADSC (IPADSC).

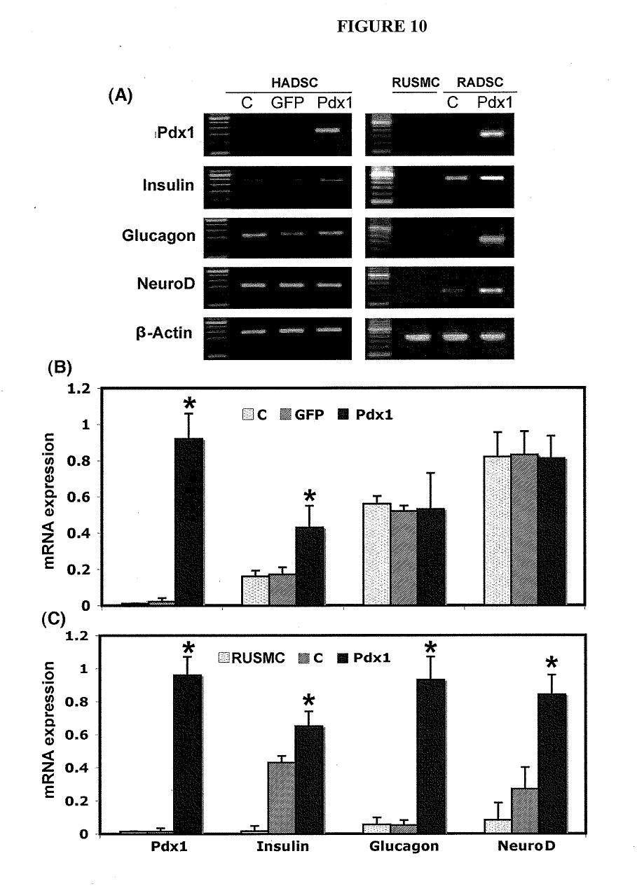

Pdx1 controls the expression of several key genes during pancreatic development, including insulin, glucagon and NeuroD genes. RT-PCR analysis showed that human ADSC expressed glucagon and NeuroD constitutively (FIG. 10). They also expressed low levels of insulin, which were upregulated in IPADSC. Control rat ADSC expressed insulin, glucagon, and NeuroD at lower levels than IPADSC did. The specificity of expression of these three genes in control and IPADSC was confirmed by the lack of such expression in rat urethral smooth muscle cells (FIG. 10).

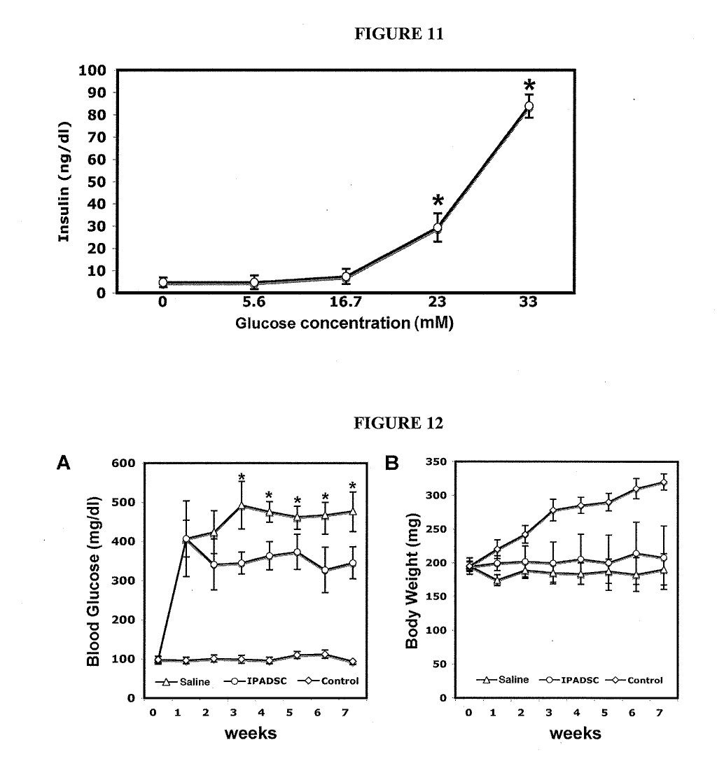

Moreover, the amount of insulin produced by IPADSC increases with increasing concentrations of glucose. As mentioned above, Pdx1 transduction resulted in the generation of IPADSC, which released approximately 30 ng/dl of insulin into the culture medium (FIG. 9). Quantitative analysis showed that these cells produced increasing levels of insulin in response to increasing concentrations of glucose (FIG. 11).

IX. Treatment of Diabetes with IPADSC

Type I diabetes rats were established by intraperitoneal injection of streptozotocin (STZ). One week after STZ injection, these rats had blood glucose levels in the range of 300 to 400 mg/dl, while control rats injected with citrate buffer only had normal blood glucose levels. Ten of the STZ-treated rats were subsequently treated with IPADSC while the other 10 STZ-treated rats were treated with saline. Treatment was done by transplantation of approximately 2 million rat IPADSC or injection of saline under renal capsule.

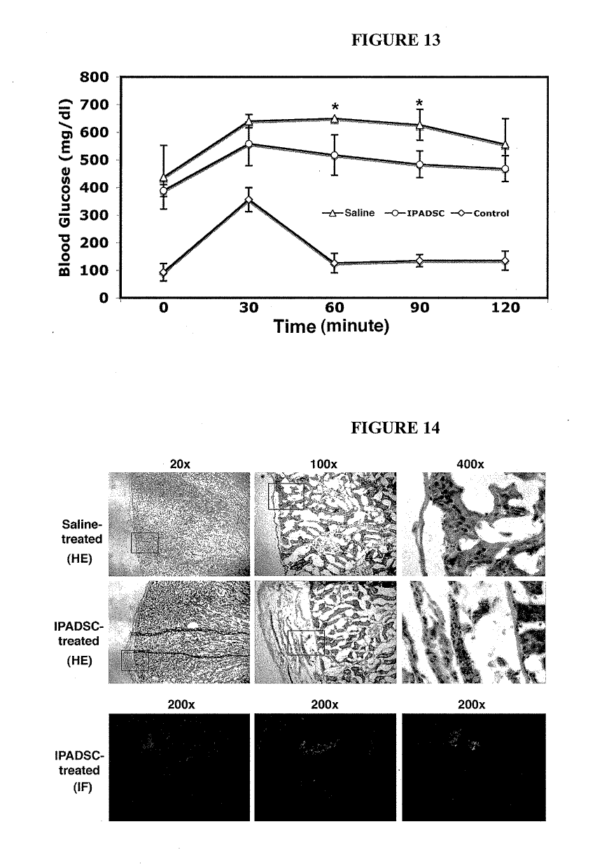

Fasting blood glucose levels and body weight were monitored weekly for 7 weeks. As shown in FIG. 12A, throughout the entire course, IPADSC-treated rats had lower blood glucose levels than saline-treated rats (P<0.05). Body weights of IPADSC-treated rats were also better than those of saline-treated rats although the difference was not statistically significant (P>0.05) (FIG. 12B). At the end of the 7^{th} week, all rats were examined for fur appearance and extent of cataract, tested for glucose tolerance, and then sacrificed for histological assessment. The results showed that IPADSC-treated rats had healthier-looking (less scruffy) fur and lesser extent of cataract than saline-treated rats. IPADSC-treated rats also had higher levels of glucose tolerance (FIG. 13). Finally, histological examination of the transplanted kidneys showed the presence of transplanted cells, which were stained positive for insulin (FIG. 14).

X. Labeling and Tracking ADSC with EdU

Currently the method of choice to label dividing cells is the incorporation of the thymidine analogue, 5-bromo-2-deoxyuridine (BrdU), into the DNA of S-phase cells. After fixation of the labeled cells, BrdU is detected with a BrdU-specific antibody. However, BrdU immunochemistry can be problematic because strong DNA denaturing conditions, such as strong acids and heating, are required to reveal the epitope, which is masked within the DNA. This introduces significant variability within and between experiments.

In an effort to overcome these problems, we sought to confirm the accuracy of an alternative thymidine analogue, 5-ethynyl-2-deoxyuridine (EdU). The terminal alkyne group of EdU allows detection using a fluorescent azide that covalently binds to the alkyne group. This detection method is fast and specific and does not require DNA denaturation. The aim of the present study was to investigate the feasibility of using EdU for labeling ADSC in vitro and for tracking the labeled cells in vivo.

A total of 12 pregnant three-month-old nulliparous Sprague-Dawley rats (Charles River Laboratories, Wilmington, Mass.) were randomly divided into two groups. One day before delivery, 200 μg EdU in PBS were injected (i.p.) in the test group and PBS only in the control group. The newborn rats were used to track the EdU labeling in vivo. One week after the delivery, adipose tissues were harvested from the adult rats for the isolation of ADSC, which were subsequently used for tracking EdU-labeled cells in vitro.

For EdU labeling, ADSC were grown on glass coverslips in DMEM supplemented with 10% bovine calf serum, penicillin, and streptomycin. For dosage effect, EdU was added to the culture media at 0 uM, 10 uM, 20 uM and 50 uM. Twenty-four h later, cells were washed with PBS followed by addition of regular culture media. For time-course study, ADSC were labeled with 10 uM EdU and then split at 1 day, 4 days, 7 days, 14 days and 21 days.

For EdU staining, after methanol fixation, cells were washed twice with PBS and then incubated in 3% BSA in PBS followed by 0.5% Triton® X-100 in PBS for 20 minutes at room temperature. The cells were then incubated with freshly made Click-it reaction cocktail (Invitrogen) for 30 minutes at room temperature in the dark. Cells were counterstained with DAPI, mounted in standard mounting media and imaged by fluorescence microscopy.

Cellular location was identified by DAPI staining of the nucleus (blue). EdU was detected by Alexa 568 (red). The results of the dosage study showed that approximately 50% of cells were EdU-labeled regardless of EdU concentration (P>0.05). In the time-course study, ADSC were labeled with 10 uM EdU and then split at 1 day, 4 days, 7 days, 14 days, and 21 days. The results showed that EdU signal in the positively labeled cells decreased with time (P<0.01).

Pregnant rats were injected i.p. with 200 ug EdU in PBS and the newborn rats were used for the harvest of various tissues at 2 h, 1 wk and 6 wk after birth. The harvested tissues were fixed in cold 2% formaldehyde and 0.002% saturated picric acid in 0.1 M phosphate buffer, pH 8.0, for 4 h followed by overnight immersion in buffer containing 30% sucrose. The specimens were then embedded in OCT Compound (Sakura Finetic USA, Torrance, Calif.) and stored at −70° C. until use. Thirty different tissues (Table 3) from both the EdU-injected group and the PBS-injected group were processed for tissue array. Fixed frozen tissue specimens were cut at 10 microns, mounted onto SuperFrost-Plus charged slides (Fisher Scientific, Pittsburgh, Pa.) and air dried for 5 min. EdU staining of tissues was performed as described above. The tissues were also stained with hematoxylin and eosin (HE staining) for general histological examination.

TABLE 3
Tissues observed for EdU staining

| | 1 | 2 | 3 | 4 | 5 | 6 | 7 | 8 | 9 | 10 |
|---|---|---|---|---|---|---|---|---|---|---|
| A | Lung | Ovary | Pancreas | Bowel | Heart | Skin | Foreskin | Stomach | Kidney | Bladder |
| B | Lung | Ovary | Pancreas | Bowel | Heart | Skin | Foreskin | Stomach | Kidney | Bladder |
| C | Muscle | Liver | Thymus | Spleen | Brain | Testis | Arm | Penis | Urethra | Fat |
| D | Muscle | Liver | Thymus | Spleen | Brain | Testis | Arm | Penis | Urethra | Fat |
| E | Bladder | Kidney | Penis | Urethra | Testis | Ovary | Stomach | Bowel | Fat | Brain |
| F | Bladder | Kidney | Penis | Urethra | Testis | Ovary | Stomach | Bowel | Fat | Brain |

Note:
A, C, E: EdU injected;
B, D, F: PBS injected

We then tracked rat ADSC movement in vivo. A total 1×10⁶ rat ADSC were labeled with 10 uM EdU for 12 hr and injected autologously to the bladder neck. The tissues were harvested at 1 d, 2 d, 1 wk, and 4 wk post-transplantation. Tissue samples were fixed in cold 2% formaldehyde and 0.002% saturated picric acid in 0.1 M phosphate buffer, pH 8.0, for 4 hours followed by overnight immersion in buffer containing 30% sucrose. The specimens were then embedded in OCT Compound (Sakura Finetic USA, Torrance, Calif.) and stored at −70° C. until use. Fixed frozen tissue specimens were cut at 10 microns, mounted onto SuperFrost-Plus charged slides (Fisher Scientific, Pittsburgh, Pa.) and air dried for 5 min. For immunofluorescence examination, the slides were placed in 0.3% H₂O₂/methanol for 10 min, washed twice in PBS for 5 min and incubated with 3% horse serum in PBS/0.3% Triton X-100 for 30 min at room temperature. After draining this solution from the tissue section, the slides were incubated at room temperature with anti-alpha smooth muscle actin antibody (Abcam Inc., Cambridge, Mass., 1:500) for 1.5 h. Control tissue sections were similarly prepared except no primary antibody was added. After rinses, the sections were incubated with FITC-conjugated secondary antibody (Jackson ImmunoResearch Laboratories, West Grove, Pa.). After washing with PBS, the slides were then incubated with fresh made Click-it reaction cocktail for 30 minutes at room temperature without light followed by staining with 4′,6-diamidino-2-phenylindole (DAPI, for nuclear staining, 1 ug/ml, Sigma-Aldrich, St. Louis, Mo.).

Four weeks after injection, tissue sections were examined by for EdU (red), alpha-smooth muscle actin (SMA, green), and nucleus (blue). EdU-labeled cells were seen in the bladder neck. Most EdU-labeled cells were localized in the connective tissue. A few EdU-labeled cells appeared to have differentiated into smooth muscle cells.

### XI. Treating Voiding Dysfunction

The experimental model most often used to assess bladder complications of diabetes is the streptozotocin (STZ)-rat model, which is a model for insulin-dependent (type 1) diabetes. The subtype of diabetes most often affecting the US population today is Type II (“adult onset”) diabetes, which is characterized by insidious onset, older age, obesity, and hypertension. The key limitation of any model in which diabetes is induced (such as the STZ model) is that the diabetes is not naturally developed and thus lacks co-morbidities such as obesity, hypertension, hyperlipidemia etc.

The Zucker fatty diabetic (ZDF) rat is a laboratory-bred rat that is genetically predisposed to develop adult-onset, type II diabetes. As the animals age, their glucose metabolism slowly deteriorates and they become progressively hyperglycemic. Furthermore, they develop the associated co-morbidities normally present in humans affected with type II diabetes: hypertension, obesity, hyperlipidemia etc. These rats are obese, mild hypertensive and continue to gain weight up to 19 weeks and then maintained through 30 weeks. In short, the ZDF rat is a more appropriate and natural animal model to study the development and course of medical illnesses that develop as consequences of poorly controlled adult-onset diabetes, e.g., diabetic voiding dysfunction (DVD).

We first refined voiding studies in the rats by developing a comprehensive urodynamic test (CUT), consisting of a 24-hour metabolic cage voiding study, followed by 4-channel awake cystometry, monitoring bladder pressure, abdominal pressure, true detrusor pressure and voiding volume. The test is completed with a high-resolution (7.5 mHz) bladder ultrasound to detect and quantitate post-void residual urine. This enabled us to detect the spectrum of voiding dysfunction (e.g., frequency, overactive bladder, decreased contractility, urinary retention etc.) occurring in ZDF rats that is typical in human DVD.

The rat's voiding function was assessed in both diabetic and non-diabetic ZDF rats. Results showed that if the rats' blood glucose levels were controlled long-term, despite being genetically predisposed to type-II diabetes (as many humans are), the rats did not develop the DVD as seen in their counterparts with poorly controlled type II diabetes. Thus, the female ZDF rat is an excellent model for studying the pathophysiology of DVD.

Fat tissue is abundant, thus, sufficient numbers of ADSC can be isolated from a person (or rat) and injected back into the same person. The procedure, known as autologous cell treatment, has the added advantage of preventing tissue rejection. ADSCs were isolated from the stromal vascular fraction (SVF) of fat tissues of humans, rats, and mice. With this strategy in mind, we divided early (10 weeks after developing diabetes) diabetic ZDF rats into two groups: one received injection of ADSC and the other received normal saline injection into the bladder wall. We then conducted urodynamic tests to assess bladder stability and urinary profile at 10 weeks. We found that injection of autologous ADSC into the bladder wall of ZDF rats reversed the OAB, while the other (control) group deteriorated to overflow urinary incontinence. Thus, the data show that injection of ADSCs into the bladder can prevent or favorably modify the development of end-stage diabetic cystopathy.

XII. Treatment of Urinary Incontinence with Syngeneic ADSC

### Methods

Twenty-two two-month-old primiparous Sprague-Dawley rats at gestational day 16 were used in this experiment. They were randomly divided into a control group (n=10) and an ADSC-transplanted group (n=12). After parturition, all rats underwent balloon dilation of the vagina and ovariectomy. One week later, the rats received injection of ADSC or PBS.

Syngeneic rat ADSC were labeled with 10 uM EdU for 12 hr prior to injection. In the treatment group, 1×10⁶ EdU labeled ADSC in 400 ul PBS were injected into the bladder neck and paraurethral tissues. In the control group, 400 ul PBS was injected into the same areas.

Four weeks after injection, all animals underwent assessment of bladder function by conscious cystometry. Cystometry results were classified as “abnormal” if bladder filling was accompanied by frequent, low volume bladder contractions with urethral leakage. Following cystometry, all animals were euthanized. The urethra, vagina, pelvic floor tissue, and bladder were harvested. Immunofluorescent staining of these tissues was performed to localize EdU, alpha smooth muscle actin (SMA), and the nuclei (DAPI staining). Chemical staining was also performed to assess differences in elastic fibers between treatment groups. Statistical analysis was done with Student's t-test.

### Results

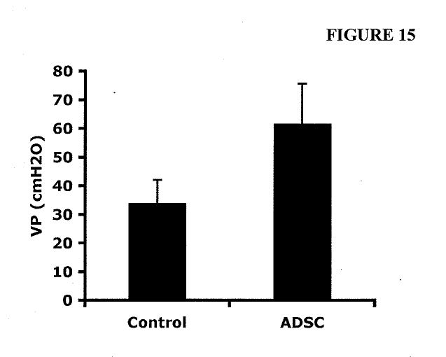

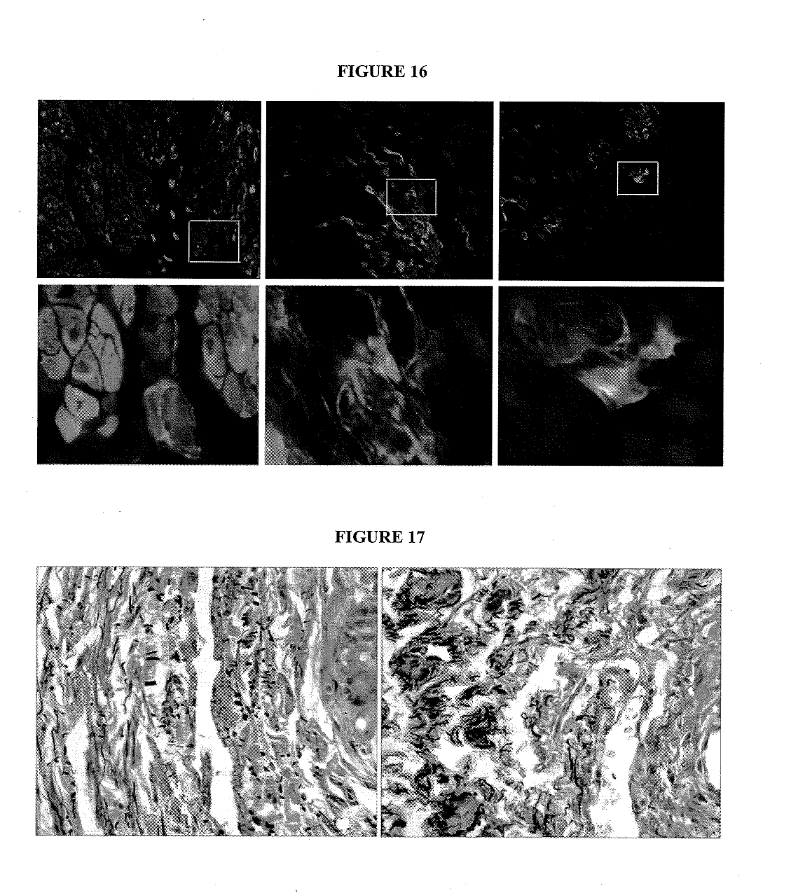

Based on cystometric criteria described above, eight out of ten (80%) rats in the control group had abnormal urinary function whereas four out of 12 (33.3%) rats in the ADSC treatment group had abnormal urinary function. Mean voiding pressure was significantly higher in the ADSC transplanted group than in the control group (61.7±13.9 cm H2O vs. 34±8 cm H2O, respectively) (P<0.05) (FIG. 15). EdU-labeled cells were identified in the submucosa of bladder neck and urethra. Some of these EdU-positive cells were also positive for SMA, suggesting differentiation of ADSC into smooth muscle cells (FIG. 16). There were significantly more elastic fibers in the urethra of rats treated with ADSC (FIG. 17).

The simulated birth injury model resulted in abnormal lower urinary tract function in a substantial number of animals. Transplanted ADSC survived in the bladder neck and paraurethral tissues and a few of them differentiated into smooth muscle cells. Animals treated with ADSC by local injection were less likely to manifest abnormal lower urinary tract function. ADSC transplantation thus holds promise as a cell-based therapy for SUI.

### XIII. Treating Erectile Dysfunction in Diabetic Rats

Bilateral crush injury of the cavernous nerves in rats is the most reliable and consistent model for study of cavernous nerve injury and regeneration. However, since selective degeneration of nitrergic nerve has been reported in rats with diabetes mellitus, we also studied erectile function and noted about 90% of type II diabetic ZDF rats became impotent 10 weeks after developing diabetes.

In rats whose cavernous nerves were crushed we injected ADSC or culture medium (control group) into the corpus cavernosum. Four weeks later, electrostimulation of the cavernous nerve was performed and the ADSC-treated group had statistically significant recovery of erectile function as compared to the vehicle treated group.

In a separate experiment, twenty ZDF type II diabetic, impotent rats were divided into two groups: one underwent injection of autologous ADSC into the corpus cavernosum of the penis and the other received injection of the cultured medium. Three weeks later the erectile function was again assessed with electrostimulation of the cavernous nerve and the ADSC treated rats had significantly better erections than the vehicle-treated group (p<0.05).

Twenty-two 10-week old ZDF type II diabetic rats (Charles River Labs) were used for experiments when they became diabetic at the age of 22 weeks. Surgery was performed under 2-3% isoflurane anesthesia. Isothermia was maintained at 37 C with a heating pad. Using a low abdominal midline incision, the right major pelvic ganglion was exposed, and the ipsilateral CN was identified. Next, the right base of the penis was exposed and the right corporal body was cannulated with a 23-G butterfly needle primed with 250 U/ml heparin-saline solution and connected to a pressure transducer (Utah Medical Products, Midvale, Utah). A bipolar stainless steel hook electrode was used to directly stimulate the right cavernosal nerve. A signal generator (National Instruments) generated monophasic rectangular pulses. Stimulus parameters were 1.5 mA, 20 Hz, pulse-width 0.2 ms, and duration 50 seconds. The intracavernous pressure (ICP) was recorded at a rate of 10 samples/s using a sensor input module (model SCXI 1121, National Instruments, Austin, Tex., USA) connected to a computer with LabView 6.0 software (National Instruments, USA). Maximum ICP was recorded.

Impotent animals were assigned to control or ADSC treatment groups. Aortic blood pressure was measured and systemic blood pressure was calculated [diastolic BP+⅓(systolic−diastolic)]. Animals with baseline ICP>60 cm were excluded from the study. Approximately 5 grams paragonadal adipose was harvested from each of the remaining rages for ADSC isolation

Seven days later, 1×10⁶ ADSC, suspended in 500 μL sterile PBS, were injected autologously into each treatment-group animal using a 31 G needle. Control animals received injection of 500 μL of PBS. Gentle tourniquet pressure was applied to the proximal base of the penis immediately before injection, and maintained for 90 seconds after injection. A single 6-0 nylon suture was placed to close and mark the injection site.

Blood glucose levels and body weight steadily increased in all animals between age 10 and 23 weeks. At 22 weeks, two of 22 (10%) animals demonstrated potency (stimulation ICP increase >90 cmH2O) during unilateral CN stimulation, and were excluded from the study. The remaining 20 animals all demonstrated a stimulation ICP increase of less than 60 cmH20, and were randomly divided into ADSC treatment (N=10) and sham control (N=10) groups. Mean baseline ICP increase age for both study groups were not significantly different (p=0.36)

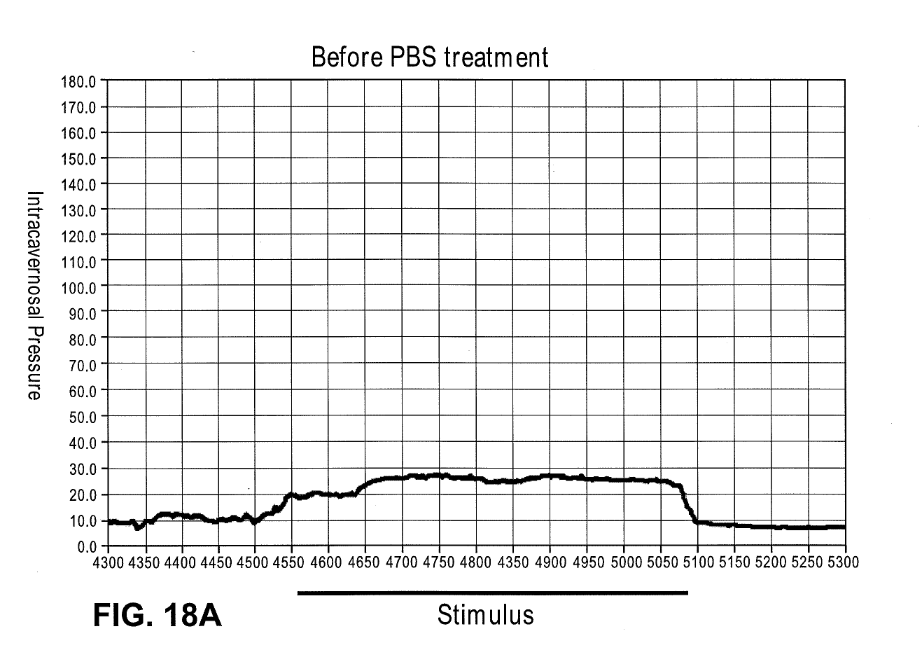

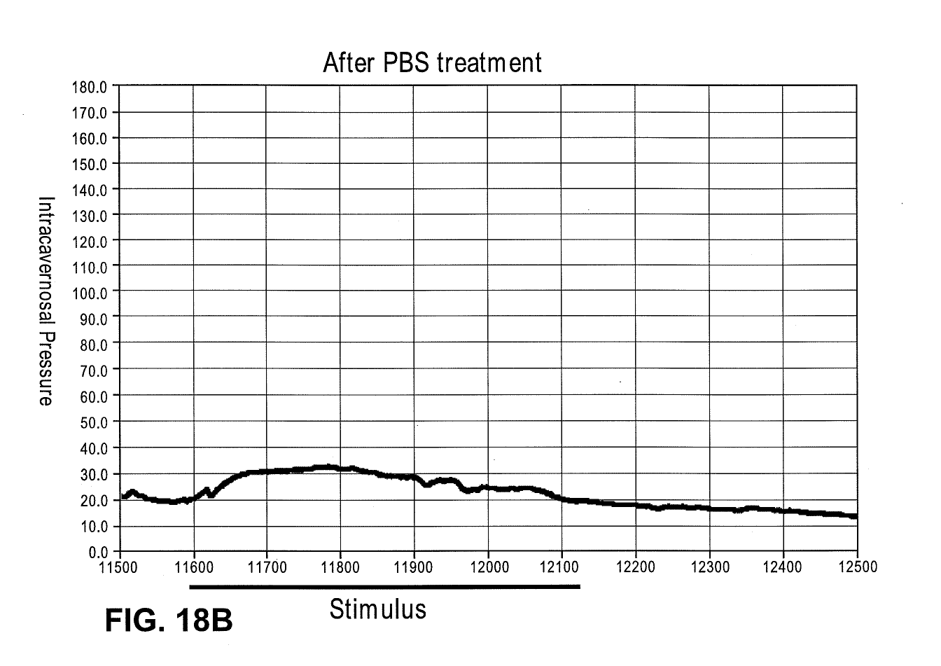

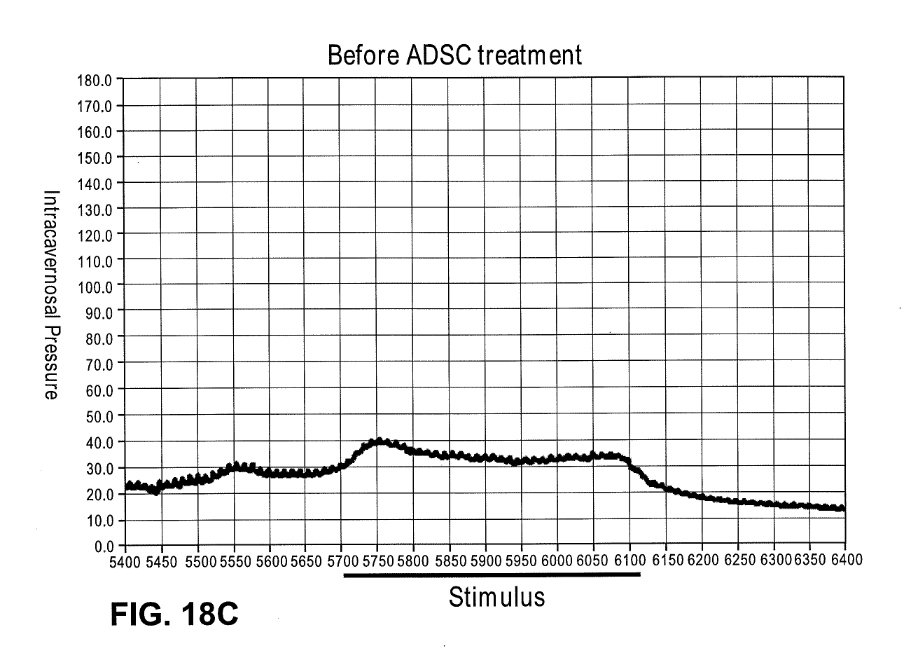

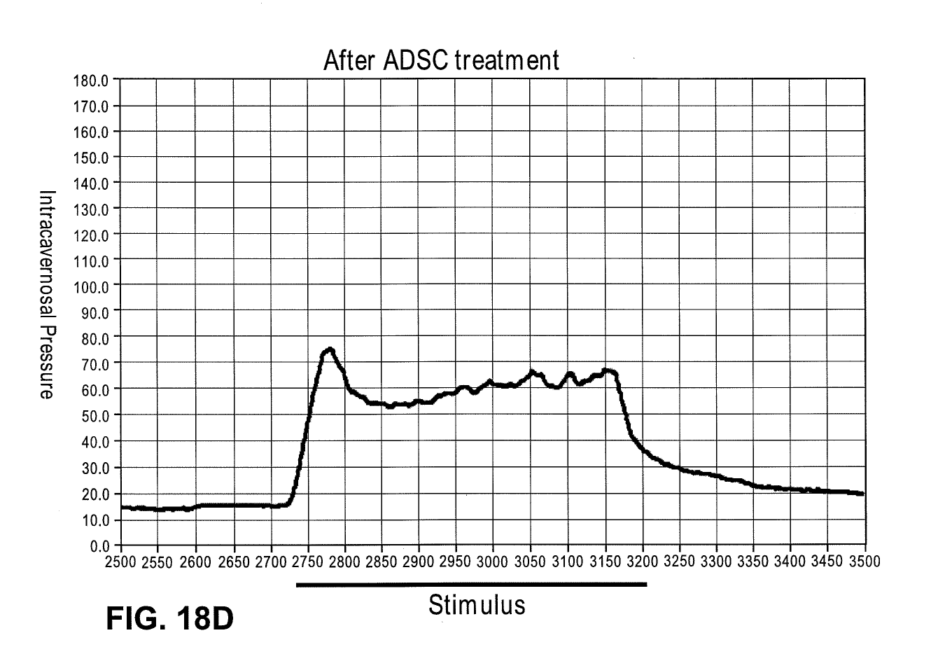

In the control group, the mean ICP after treatment (with PBS) was slightly lower than before treatment (31.4±24.3 cmH2O vs. 33.8±15.9 cmH2O). In the ADSC-treated group, the mean ICP increased significantly (P<0.002) to 65.3±15.4 cmH2O from the pre-treatment value of 27.4±15.4 cmH2O (FIG. 18). Mean systemic pressure between control (126.7±19.6 cmH2O) and treatment groups (119.4±9.2 cmH2O) were not significantly different (P=0.301).

### XIV. Endothelial Differentiation of ADSC

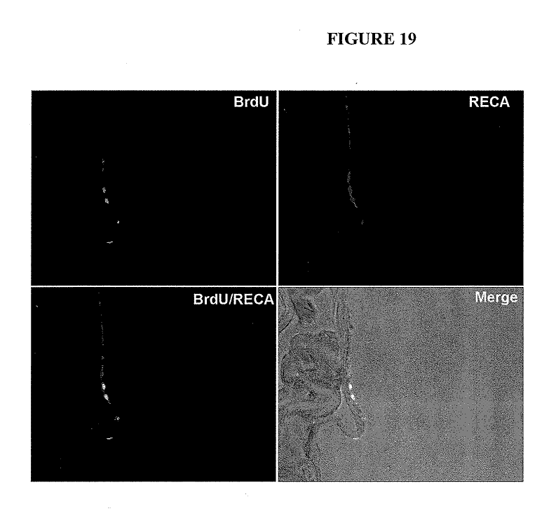

To test whether ADSC could differentiate into endothelial cells, we injected ADSC into the penis of rats and examined the tissue 4 weeks later. ADSC were identified by BrdU staining, and approximately 5% of them also stained positive for rat endothelial cell antigen (RECA-1). These cells were localized to the sinusoid endothelium as revealed by the superimposed images of fluorescence and phase-contrast microscopy (FIG. 19).

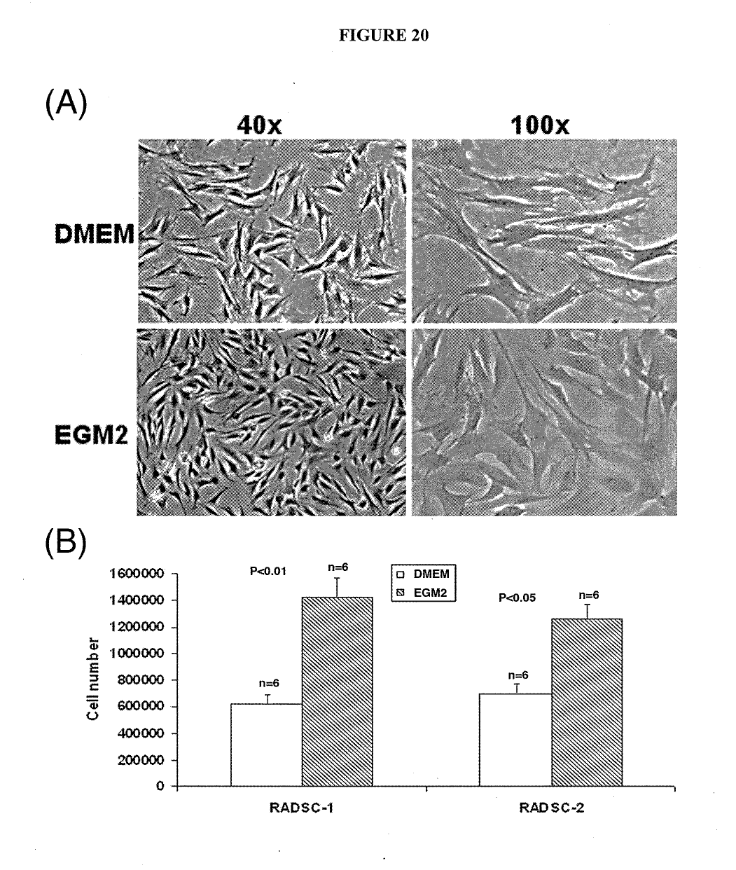

The majority of published ADSC studies describe culturing ADSC in DMEM. We also routinely cultured endothelial cells in EGM2, which is a commercially available endothelial growth medium. When DMEM in ADSC cultures was replaced with EGM2, the cells reached confluence faster and appeared more compact (larger nuclei) than cells that remained in DMEM. A proliferation assay confirmed that ADSC grew much more rapidly in EGM2 than in DMEM (FIG. 20).

We next determined expression of endothelial markers in ADSC grown in EGM2. RADSC-1 and RADSC-2 were grown in DMEM (un-induced) or EGM2 (induced) and stained for endothelial markers CD31, vWF, and eNOS. Immunocytochemistry showed that ADSC grown in EGM2 expressed all three endothelial specific markers. Matrigel tube formation assay also showed that ADSC grown in EGM2 were able to form endothelial-like tube structures. Additionally, LDL uptake assay showed that ADSC grown in EGM2 were capable of LDL uptake. The endothelial specificity of these three assays was supported by positive results with HUVEC cells. Human umbilical vein endothelial cells (HUVEC) served as positive control. Experiments were repeated 3 times. These cells can thus be called Endothelial-like Adipose tissue Derived Stem Cells (EADSC).

We next tested whether ADSC differentiation was reversible by replacing EGM2 with DMEM. Culturing in DMEM for ten days resulted in the disappearance of all endothelial characteristics. We then reintroduced EGM2 to the cells for six days, and this resulted in the reappearance of all endothelial characteristics, albeit at reduced levels. These tests established that the EGM2 medium contains specific factors capable of inducing ADSC endothelial differentiation.

### XV. Endothelial Inducing Factors

EGM2 medium is supplied by the manufacturer in the form of a basal medium (EBM2) and individual vials of supplemental factors. This packaging format allowed us to test the importance of each supplemental factor as related to ADSC endothelial differentiation. Specifically, we prepared “subtraction” EGM2 media by omitting one supplemental factor at a time. We then maintained ADSC in each subtracted EGM2 medium for one week and then assayed for their LDL-uptake ability, which has been shown to be the most reliable endothelial marker.

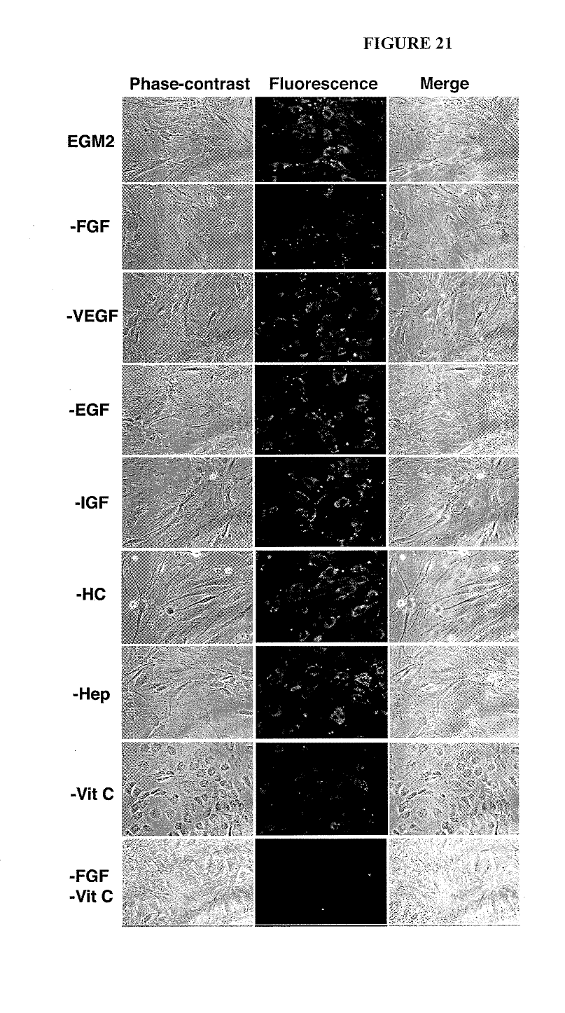

The results show that, among growth factors, the omission of VEGF, EGF, or IGF had essentially no effect, whereas the omission of FGF2 greatly diminished LDL-uptake ability (FIG. 21). Among non-growth factors, the omission of hydrocortisone or heparin had essentially no effect, whereas the omission of vitamin C greatly diminished LDL-uptake ability. When both FGF2 and vitamin C were omitted, ADSC exhibited essentially no LDL-uptake ability.

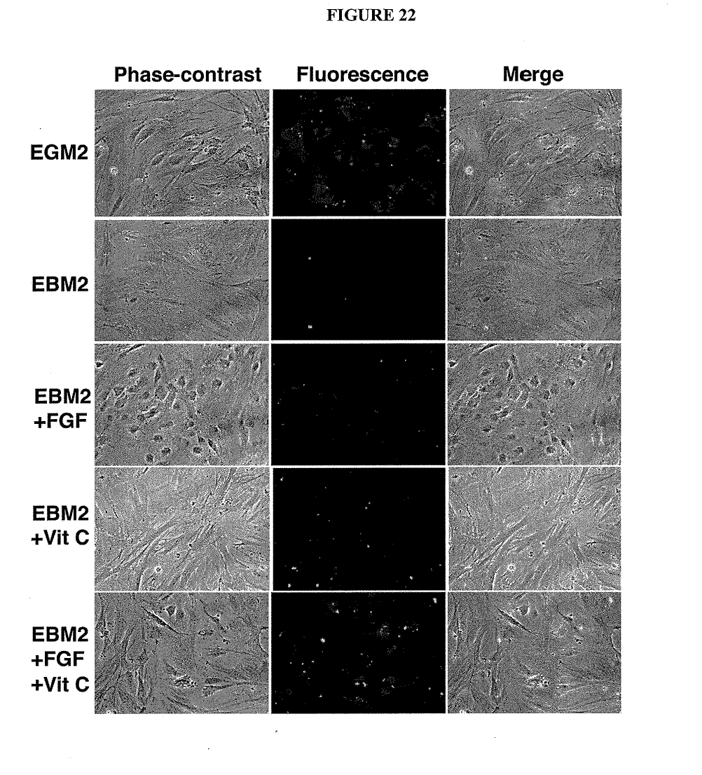

To further confirm the importance of FGF2 and vitamin C, we prepared “addition” media by adding FGF2 and/or vitamin C to EBM2, maintained ADSC in these media for one week, and then assayed ADSC's LDL-uptake ability. The results show that (1) EBM2 supplemented with FGF2 and vitamin C was nearly as effective as EGM2, (2) EBM2 supplemented with FGF2 was still effective, albeit at a reduced level, and (3) EBM2 supplemented with vitamin C was still somewhat effective, but at a much reduced level (FIG. 22).

The above experiments identified FGF2 as the only growth factor required for the induction of ADSC's LDL-uptake ability. We then tested whether FGF2 was able to induce the expression of additional endothelial characteristics. RADSC-1 cells were grown in DMEM, EGM2, or EBM2 supplemented with FGF2 and vitamin C. They were then stained for endothelial markers CD31, vWF, and eNOS, and tested for tube formation. The results showed that cells grown in FGF2/vitamin C-supplemented EBM2 acquired all of the tested endothelial markers, as did cells grown in the completely supplemented EGM2.

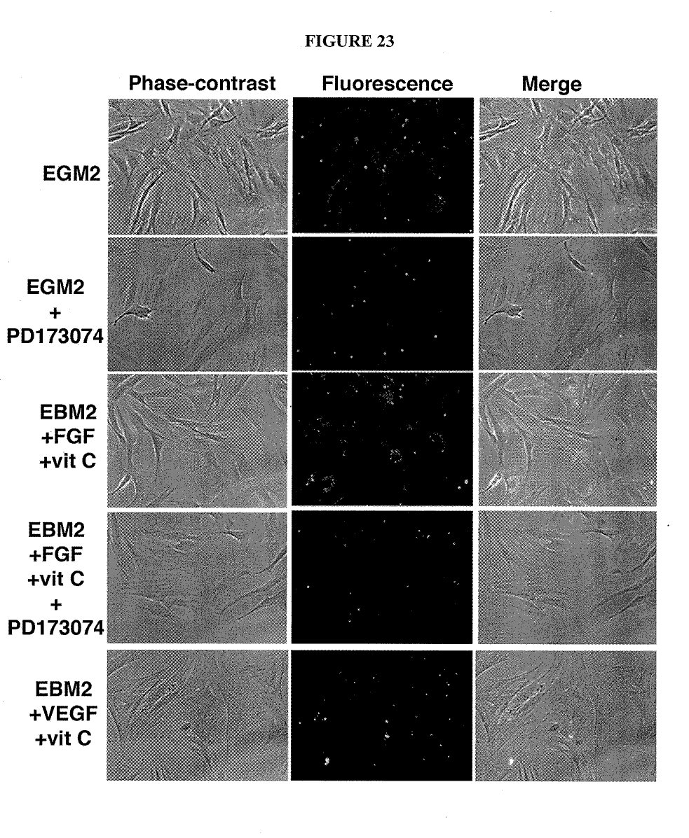

To further confirm the critical role of FGF2 in ADSC endothelial differentiation, we conducted ADSC differentiation experiments in the presence or absence of PD173074, a selective inhibitor for FGF receptor (FGFR1). In the absence of PD173074 (with the addition of solvent only), cells grown in either the completely supplemented EGM2 or the FGF2/vitamin C-supplemented EBM2 acquired the LDL-uptake ability (FIG. 23). In the presence of PD173074, cells grown in either medium were unable to do so (FIG. 23). To ensure that VEGF signaling did not interfere with this test, as PD173074 is known to have a weaker inhibitory action on VEGF receptor (VEGFR2), we showed that ADSC grown in VEGF/vitamin C-supplemented EBM2 did not acquire the LDL-uptake ability.

XVI. Prevention of Type II Diabetic Peripheral Neuropathy with Injection of Syngenic ADSC

One common complication of chronic, uncontrolled diabetes, is peripheral neuropathy, characterized by decreased sensation. Decreased peripheral sensation leads to increased peripheral injuries, as the subject cannot feel when he/she has injured the extremity. They are less apt to withdraw the limb from pain, and/or, they are less apt to discover the injury, owing to lack of complete sensation. Injuries become chronic, and ultimately infected. The additional effect of poor wound healing, from diabetes, accelerates the path toward uncontrolled infection at the injury site. Ultimately, many diabetics require amputation of injured extremities.

Randall-Selito or Randall-Selito/Ugo Basile test has been validated in STZ-induced type I diabetic animals to quantify the sensation threshold in an extremity (foot or paw). This is also called the “nociceptive mechanical flexion reflex,” and can be quantified with an Ugo Basile Analgesymeter® (Stoelting, Chicago, Ill., USA), which applies a linearly increasing mechanical force to the dorsum of the rat's hind paw.

Another validated measure of neuropathic sensory loss is the Von Frei hair test, in which a fine mechanical force is applied the animal's ventral paw surface. When the animal “feels” the hair filament, it reflexively pulls its paw away. The minimum force associated with a pulling-away response is measured with this test. The test starts with the lowest mechanical force, and is repeated with increasing force. A lower mechanical force is associated with better/more normal sensation.

ADSCs appear to improve tissue health by increasing local tissue vascularity and nerve-end budding (nerve growth) as well as decreasing local tissue apoptosis (decreased caspace). We sought to determine whether ADSCs can help restore peripheral cutaneous (“touch and pressure”) sensation, and improve pathologic hyperalgesia, in a type-II diabetic animal model.

Groups of obese male type II diabetic rats, and male ZDF lean control rats (non-obese, mildly diabetic) were assessed at baseline. Each underwent measurement of rear (right and left) paw sensation threshold, using the Ugo Basile Analgesymeter® and the Von Frei Hair test. These serve as baseline measurements. The animals were then randomly assigned to treatment and control groups. Treatment group animals underwent injection of 2.5 million ADSCs into the penis. The control group diabetic animals (positive controls) underwent no procedures.

A second control group of animals—lean non-obese mildly diabetic ZDF male rats (blood glucose ˜225) served as negative controls. Six weeks after treatment, all animals underwent repeat Von Frei Hair and Randal Selito/Ugo Basile tests.

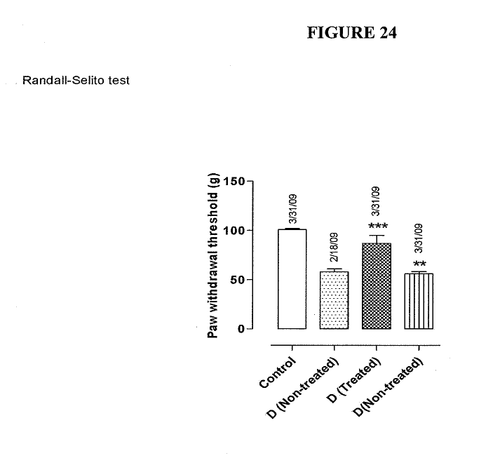

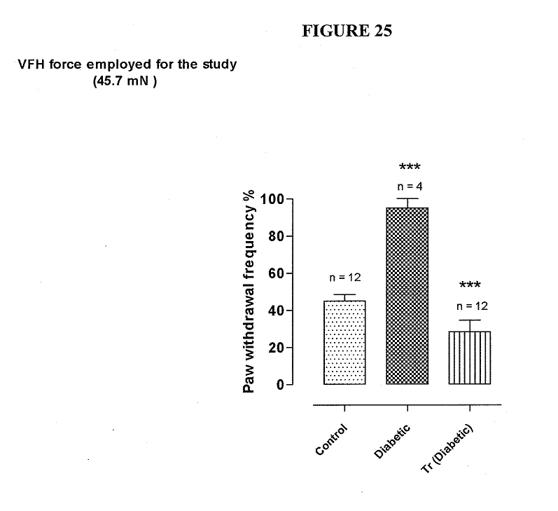

The treated animals had significantly more normal sensation in both tests (see FIGS. 24 and 25). Both the lean and obese ZDF animals had baseline results suggesting significantly abnormal pain threshold, by the Randall-Selito test.

The results of the Von Frei test show that, compared to normal values for a healthy Sprague Dawley rat, as well as compared to the mildly diabetic lean ZDF control group animals, the untreated severely diabetic obese ZDF rats had highly abnormal sensation thresholds, while the ADSC treated animals had sensation threshold higher than that of the obese and lean controls, and, approached the level for a healthy rat.

It is understood that the Examples and embodiments described herein are for illustrative purposes only and that various modifications or changes in light thereof will be suggested to persons skilled in the art and are to be included within the spirit and purview of this application and scope of the appended claims. Any publications cited herein are incorporated by references for all purposes, except to the extent they conflict with the present disclosure.

What is claimed is:

1. A method of generating Insulin-Producing Adipose tissue Derived Stem Cells (IPADSC), said method comprising
(i) isolating a population of Adipose tissue Derived Stem Cells (ADSC);
(ii) introducing an expression cassette driving expression of Pdx-1 or VP16 into said population of ADSC,
thereby generating IPADSC.

2. The method of claim 1, further comprising introducing said IPADSC into a subject.

3. The method of claim 2, wherein said subject has diabetes, or a predisposition for diabetes.

4. A method of treating or preventing diabetes in a subject, said method comprising administering a composition comprising Insulin-Producing Adipose tissue Derived Stem Cells (IPADSC) to the subject, thereby treating or preventing diabetes in the suject.

5. The method of claim 4, wherein said subject has type 1 diabetes.

6. The method of claim 4, wherein the administering is by injection.

7. A method of generating Endothelial-like Adipose tissue Derived Stem Cells (EADSC), said method comprising contacting Adipose tissue Derived Stem Cells (ADSC) with media comprising Fibroblast Growth Factor 2 (FGF2).

8. The method of claim 7, wherein said media further comprises Vitamin C.

9. A method for treating or preventing an ischemia-related disorder in a subject, comprising:
(i) contacting Adipose tissue Derived Stem Cells (ADSC) with media comprising Fibroblast Growth Factor 2 (FGF2), such that the ADSC are transformed into Endothelial-like Adipose tissue Derived Stem Cells (EADSC); and
(ii) administering a composition comprising said EADSC to the subject, thereby treating or preventing the ischemia-related disorder in the subject.

10. The method of claim 9, wherein the media further comprises Vitamin C.

11. The method of claim 9, wherein the ischemia-related disorder is selected from the group consisting of stroke, angina, bed ulcer, foot ulcer, intermittent claudication and kidney disease.

12. A method of treating or preventing peripheral neuropathy in a subject, said method comprising administering a composition comprising Adipose tissue Derived Stem Cells (ADSC) to the subject, thereby treating or preventing peripheral neuropathy in the subject.

13. The method of claim 12, wherein the subject has diabetes.

14. A method for treating urinary tract injury or incontinence in a subject, said method comprising administering a composition comprising Adipose tissue Derived Stem Cells (ADSC) to the bladder or urethra of the subject, thereby treating urinary tract injury or incontinence in the subject.

15. A method for treating erectile dysfunction in a subject, said method comprising administering a composition comprising Adipose tissue Derived Stem Cells (ADSC) to the penis of the subject, thereby treating erectile dysfunction in the subject.

16. A method for treating a wound in a subject, said method comprising administering a composition comprising Adipose tissue Derived Stem Cells (ADSC) to the wound area, thereby treating the wound in the subject.

17. A method for isolating Adipose tissue Derived Stem Cells (ADSC), the method comprising the steps of:
(i) incubating a solution containing adipose tissue and collagenase;
(ii) separating the lipids, collagenase and adipose cells in the incubated solution into layers by subjecting the incubated solution to centrifugation;
(iii) isolating and filtering the collagenase layer;
(iv) subjecting the filtered collagenase layer to a second centrifugation step, thereby obtaining a preparation of recycled collagenase; and
(v) treat fresh whole-resected adipose tissue, or liposuction-resected (lipoaspirate) with said recycled collagenase.

18. A method for preserving a population of Adipose tissue Derived Stem Cells (ADSC), the method comprising the steps of:
(i) combining said population with a preservation solution comprising superoxide dismutase at a concentration of at least 5,000 U/L; and
(ii) mixing said solution with said sample.

19. A composition comprising Adipose tissue Derived Stem Cells (ADSC); and (ii) superoxide dismutase, wherein said superoxide dismutase is present at a concentration of at least 5,000 U/L.

20. A method for isolating a subpopulation of multipotent Adipose tissue Derived Stem Cells (ADSC), the method comprising the steps of:
(i) contacting a starting population of ADSC with a set of antibodies, wherein said set of antibodies comprises anti-CD34, anti-CD90, and anti-SSEA1 antibodies;
(ii) identifying the ADSC which express the antigens recognized by said set of antibodies; and
(iii) isolating the adipose tissue-derived stem cells identified in step (ii), wherein the isolated cells constitute a subpopulation of multipotent ADSC.
